(19) United States
(12) Patent Application Publication
(10) Pub. No.: US 2015/0099012 A1
(43) Pub. Date: Apr. 9, 2015

(54) METHODS FOR ACTIVATING RETROVIRUS IN LATENT INFECTED CELLS, AND COMPOUNDS FOR USE THEREIN

(71) Applicant: Erasmus University Medical Center Rotterdam, Rotterdam (NL)

(72) Inventor: Tokameh Mahmoudi, Rotterdam (NL)

(21) Appl. No.: 14/382,413

(22) PCT Filed: Mar. 1, 2013
(86) PCT No.: PCT/NL2013/050131

§ 371 Date: Sep. 2, 2014

(30) Foreign Application Priority Data
Mar. 2, 2012 (NL) PCT/NL2012/050128

(51) Int. Cl. A61K 33/14; A61K 31/185; A61K 31/404

(52) U.S. Cl. CPC A61K 33/14; A61K 31/404; A61K 31/185
USPC 424/677; 514/414; 514/7.6; 435/375; 435/29

(57) ABSTRACT

The present invention relates to a method for increasing retrovirus transcription in an infected eukaryotic cell, comprising increasing Wnt pathway signaling in said cell such that transcription of said retrovirus is increased. Also, the present invention relates to a method of treating a subject infected with a retrovirus, said method comprising administering to said subject an activator of the Wnt pathway in an amount that increased Wnt pathway signaling in resting memory CD4+ T cells of said subject such that the retroviral long terminal repeat (LTR) in said cells is activated or de-repressed.

### FIELD OF THE INVENTION

The invention is in the field of antiviral therapy. In particular the present invention relates to curing treatments of viral infection associated with latent integration of a retrovirus in a host cell genome, in particular infection by HIV. The invention relates to methods of purging or re-activation of latent virus from host cells, to compounds for use in such methods and to methods of screening for such compounds.

### BACKGROUND OF THE INVENTION

The treatment of viral infections has benefitted greatly from recent developments in the field of antiretroviral therapeutics. However for many infections like the Human Immunodeficiency Virus (HIV) a complete cure is still lacking. Treatment for HIV infection consists of highly active antiretroviral therapy, or HAART. HAART involves combination therapy with a cocktail of several (typically three or four) antiretroviral drugs preferably selected from different classes of antiretroviral drugs targeting different stages of the HIV life cycle. Typically, these classes include nucleoside or nucleoside analogue reverse transcriptase inhibitors (NRTIs or NARTIs), protease inhibitors and non-nucleoside reverse transcriptase inhibitors (NNRTI). HAART allows the stabilization of the patient's symptoms and viremia, but it neither cures the patient, nor alleviates the symptoms. High levels of HIV, often HAART resistant, return once treatment is stopped. Thus, patients require HAART medication for the rest of their lives. The main impediment to a curative treatment for HIV infected patients is the existence of a reservoir of host cells containing replication-competent HIV. This is largely due to the pathology of HIV infection.

After host CD4+ T cell infection and nuclear entry, HIV DNA integrates into the T cell genome as a chromatin template. Through unclear mechanisms, a very small percentage of infected T cells become latent, meaning that these infected cells are not actively producing virus but retain the capacity to do so.

The effectiveness of modern HAART together with the impracticality of its current life-long treatment protocol highlight the need for new strategies towards tackling HIV, i.e. approaches based not only on replication control, but on the goal of eradicating HIV from infected patients in a feasible economic and temporal manner. Activating transcription of the LTR of latently infected cells will cause activation and replication of the virus, Activating latent virus will increase the effectiveness of HAART treatment in targeting both active and previously latent HIV infected cells.

WO2088/016994 discloses that activation of Wnt signalling potentially restricts HIV replication both in peripheral blood mononuclear cells (PBMCs) and astrocytes. Inducing Wnt signalling, leading to the activation of β-catenin by addition of lithium chloride (LiCl) inhibited replication of a number of HIV isolates in PBMCs.

Henderson and Al-Harti (J. Neuroimmune Pharmacol (2011) 6:247-259) discloses the role of β-Catenin/Wnt signaling in repression of HIV replication.

Kumar et all (J. Virol (2008) 82(6):2813-2820) describes that an active Wnt/β-catenin pathway is inhibitory for HIV virus replication. Lithium is used to activate β-catenin. On page 2817 it is disclosed that lithium inhibits reactivation of latent HIV.

Narasipura et all (J. Viol (2012) 86(4):1911-1921) is a review of the role of β-catenin in the transcription of HIV in astrocytes. It discloses that knock-down of β-catenin increased LTR activity.

Surprisingly, the present invention shows that increasing the Wnt pathway signalling actually increases retrovirus transcription in an infected eukaryotic cell, contrary to the finding of the prior art, wherein it was seen that activation of Wnt signalling represses HIV transcription.

### SUMMARY OF THE INVENTION

In a first aspect, the present invention provides a method for increasing retrovirus transcription in an infected eukaryotic cell, comprising increasing Wnt pathway signaling in said cell such that transcription of said retrovirus is increased.

In a preferred embodiment of aspects of the invention and/or embodiments thereof, the infected cell is a latently infected cell wherein retrovirus transcription is repressed, said method comprising activating said repressed retrovirus by increasing Wnt pathway signaling in said cell such that the retroviral long terminal repeat (LTR) is activated or de-repressed.

In another preferred embodiment of aspects of the invention and/or embodiments thereof, the Wnt pathway signaling in said cell is increased such that beta-catenin is stabilized and recruited to the retroviral long terminal repeat (LTR).

In yet another preferred embodiment of aspects of the invention and/or embodiments thereof, the Wnt pathway signaling in said cell is increased by contacting said cell with a Wnt pathway activator, an inhibitor of glycogen synthase kinase-3 beta (GSK-3β), an inhibitor of adenomatous polyposis coli (APC), or an inhibitor of axin, or a combination thereof. In preferred embodiments wherein a GSK-3β inhibitor is used, the GSK-3β inhibitor may be (6-bromoindirubin-3′-oxime (BIO) and/or lithium (such as lithium or lithium carbonate), and/or CHIR-99021 (6-(2-(4-(2,4-Dichlorophenyl)-5-(4-methyl-1H-imidazol-2-yl)-pyrimidin-2-ylamino)ethyl-amino)-nicotinonitrile) which is a very specific GSK-3β inhibitor. In a preferred embodiment of aspects of the present invention and/or embodiments thereof in the event lithium is used to increase Wnt pathway signalling, the amount of lithium administered is such that the therapeutic plasma concentration is between 0.1-10 mmol/L.

The Wnt pathway activator may be a Wnt receptor agonist such as a Wnt ligand, and/or β-catenin such as the constitutively active mutant S33Y β-catenin. In preferred embodiments wherein a Wnt ligand is used, the preferred Wnt ligand is Wnt3A, but the use of other Wnt ligands is envisaged herein. Alternatively, or in addition to the use of Wnt ligands, the Wnt pathway activator may be a Leucine-rich G-protein-coupled Receptor (LGR) agonist, e.g. an agonist of LGR4, LGR5 or LGR6, such as an LGR ligand. A very beneficial LGR ligand for use in aspects of this invention is R-spondin (RSpo) of which any can be used, such as for instance RSpol-4. In yet further preferred embodiments, a combination of one or more GSK-3β inhibitors with one or more Wnt pathway activators may be used.

In still further preferred embodiments of aspects of the invention and/or embodiments thereof, the method further comprises increasing histone acetylation in said cell. Preferably histone acetylation is increased by contacting said cell with a histone deacetylase inhibitor (HDACi). A highly preferred HDACi is valproic acid (VPA).

Another highly preferred embodiment in aspects of the invention and/or embodiments thereof is increasing Wnt signaling in said cell by contacting said cell with a GSK-3β inhibitor, preferably lithium, and simultaneously increasing histone acetylation in said cell by contacting said cell with a HDACi, preferably valproic acid (VPA).

In still further preferred embodiments of aspects of the invention and/or embodiments thereof, the method further comprises contacting said cell with a compound selected from a cytokine, a T cell activation signal, a macrophage activator, a protein kinase C (PKC) activator, a nuclear factor κB (NF-κB) activator, a transcription elongation inducer, and combinations thereof, preferably the compound is selected from the group consisting of LPS, 11-7, prostratin, hexamethylbisacetamide (HMBA), and Cyclin T.

In still further preferred embodiments of aspects of the invention and/or embodiments thereof, the retrovirus is the human immunodeficiency virus (HIV), preferably HIV-1.

In still further preferred embodiments of aspects of the invention and/or embodiments thereof, the LTR is the 5′-LTR.

In still further preferred embodiments of aspects of the invention and/or embodiments thereof, the cell is a CD4+ T cell, more preferably a resting memory CD4+ T cell.

In another aspect, the present invention provides a method of treating a subject infected with a retrovirus, said method comprising administering to said subject an activator of the Wnt pathway in an amount that increased Wnt pathway signaling in resting memory CD4+ T cells of said subject such that the retroviral long terminal repeat (LTR) in said cells is activated or de-repressed.

In another aspect, the present invention provides a method of treating a subject infected with a retrovirus, said method comprising performing on said subject a method for increasing retrovirus transcription in an infected cell as described above, comprising increasing Wnt pathway signaling in said cell such that transcription of said retrovirus is increased. All preferred embodiments as described for that method apply to the present method of treating a subject.

In a preferred embodiment of aspects of the present invention and/or embodiments thereof in the even lithium us used to increase Wnt pathway signalling, the amount of lithium administered is such that the therapeutic plasma concentration is between 0.1-10 mmol/L.

In a preferred embodiment of a method of treating a subject the subject receives antiretroviral therapy.

In further preferred embodiments of the method of treating a subject, the activator is selected from Wnt3A (e.g. Wnt3, Wnt5, etc), R-spondin (e.g. Rspol-4), 6-Bromoindirubin-3′-oxime (BIO), CHIR-99021, and lithium, and combinations thereof. A highly preferred method of treatment comprises the administration of the Wnt pathway activator in combination with the administration of a histone deacetylase inhibitor (HDACi), preferably valproic acid (VPA) and/or SAHA.

In another aspect, the present invention provides a method of screening for a compound that activates latent retrovirus infected cells, comprising contacting retrovirus target cells with a test compound and assessing whether said test compound activates the Wnt pathway in said cells.

In yet another aspect, the present invention provides an activator of the Wnt pathway, preferably selected from lithium and BIO, for use in the treatment of HIV infection.

In yet another aspect, the present invention provides an activator of the Wnt pathway in combination with a HDACi for use in the treatment of HIV infection.

In another aspect, the present invention provides a method to establish the therapeutic dose for a patient infected with a retrovirus, of a test compound that activates latent retrovirus infected cells, comprising contacting retrovirus target cells of said patient with a test compound and assessing whether said test compound activates the Wnt pathway in said target cells.

In yet another aspect, the present invention provides a method to monitor a patient receiving treatment with a compound that activates latent retrovirus infected cells, said method comprising contacting retrovirus target cells from said patient with said compound and assessing whether said compound activates the Wnt pathway in said target cells.

### DESCRIPTION OF THE DRAWINGS

FIG. 1 illustrates the method for generation of the CD4+ T cell line model system reflecting HIV latency.

FIG. 2 depicts the Wnt pathway and its components.

FIG. 3 shows the chromatin organization of the HIV LTR. Chromatin organization of the HIV-1 provirus demonstrate the presence of at least three precisely positioned nucleosomes, nuc-0, nuc-1 and nuc-2 and their intervening nucleosome-free regions (or DNase hypersensitive sites DHS1 and DHS2). In particular nuc-1, the nucleosome positioned immediately downstream of the transcription start site (TSS) is repressive to transcription and surrounded by two large domains of nucleosome-free DNA. Following activation, nuc-1 becomes rapidly and specifically disrupted. In the immediate-early phase of HIV infection, cellular transcription factors activate transcription from the viral promoter in the 5′-LTR, leading to accumulation of viral Tat protein, a potent transactivator, causing a Tat-dependent positive feedback loop and surge in transcription. Tat also leads to the chromatin remodeling of nuc-1, the repressive nucleosome positioned immediately downstream of TSS. Is has been reported that Tat recruits many activating cofactors including the SWI/SNF chromatin-remodeling complex to activate LTR transcription.

FIG. 4 shows the method for determining nucleosome position changes and direct binding of LEF/b-catenin at HIV LTR in latent and activated states and results of tests using that method. (A) Schematic representation of strategy to explore nucleosome position changes and enrichment of LEF/b-catenin at the HIV LTR in its repressed state and after Wnt activation. Jurkat latent cells at 0, and 12 hours after stimulation with LiCl, BIO VPA, or LiCl and VPA together were crosslinked and sonicated to yield DNA fragments of approximately 150 bp. DNA accessibility was monitored by formaldehyde-assisted isolation of regulatory elements (FAIRE) while nucleosome occupancy was determined by histone H2B Chromatin immunoprecipitations (ChIPs). To determine enrichment of LEF, b-catenin, or Histone Acetyl H3, we performed ChIPs with antibodies specific for LEF1, b-catenin, Ac H3. (B) Wnt activation by treatment with LiCl/BIO is accompanied by an increase in DNA accessibility over the DHS1 of the HIV LTR. Co-treatment of latent cells with LiCl and VPA caused an increase in DNA accessibility over the repressive positioned nuc-1(C) Wnt activation by LiCl and BIO causes a reduction in histone density over HIV DHS1 while co-treatment with LiCl and VPA caused a decrease in histone density also over the repressive positioned nuc-1 as determined by H2B ChIP, presented as percent immunoprecipitated DNA over input. FAIRE results are presented as fold change respective to the unstimulated value (normalized to 1) for each primer pair. (D) b-catenin while absent from the LTR in the repressed state, directly binds to the LTR upon LiCl/BIO activation. LEF1 binding, while present on the LTR in the repressed state is enriched upon LiCl/BIO treatment. ChIPs are presented as percent immunoprecipitated DNA over input. Immunoprecipitated DNA from ChIPs and phenol:chloroform extracted DNA from FAIRE were analyzed by qPCR using primer pairs specific for the HIV LTR nuc-0, DHS1, nuc-1, and nuc-2 regions, and control region amplifying upstream of the Axin2 gene. For all ChIP and FAIRE experiments, error bars represent the SEM of at least three independent experiments. * p<0.05, ** p<0.01.

FIG. 5 shows that Wnt pathway activation and VPA treatment synergistically purge latent HIV.

FIG. 6 shows that the activation of the Wnt pathway by exogenous expression of constitutively active S33Y β-catenin mutant activates latent HIV infected cells. A) Exogenous expression of S33Y β-catenin in S-Lat30 latent HIV infected cell line results in activation of TCF/LEF-driven TOPFLASH luciferase activity. B) Exogenous expression of S33Y β-catenin in S-Lat30 latent HIV infected cell line results in increased mRNA expression of the endogenous Wnt target genes AXIN2, ZCCHC12, TCF1, MMP2, MMP9, but not control gene GAPDH, or the NFkb target genes TLR2 and ICAM1 as determined by quantitative RT-PCR. S33Y β-catenin expression also results in a concomitant activation of latent HIV in S-Lat30 HIV infected cell line as quantitated by increased mRNA expression of HIV P24 Gag and the LTR-reporter GFP. Expression data are presented as fold induction normalized to Cyclophillin A control.

FIG. 7 shows that the treatment of latent HIV infected cell with Wnt 3A ligand activates latent HIV and endogenous Wnt target genes but not NFkB target genes. Latent HIV-infected S-Lat 30 (A) and J-LatP32 (B) were treated with Wnt3A or control conditioned media. Relative mRNA expression levels of endogenous Wnt target genes AXIN2, ZCCHC12, TCF1, MMP9, the HIV P24 Gag and the LTR-reporter GFP, control (GAPDH) and NFkB target genes were quantitated by qRT-PCR. Expression data are presented as fold induction normalized to Cyclophillin A control.

FIG. 8 shows that the treatment of latent HIV infected cells with GSK3beta inhibitor CHIR-99021 activates the latent HIV LTR. J-Lat P44, S-Lat 24, J-Lat P21, J-Lat 11.1, S-Lat 9 and S-Lat 30 latently infected Jurkat and SupT1 cell lines were treated with CHIR-99021 (1 μM) for 24 hours, and GFP expression was monitored by FACS analysis to determine activation of the latent LTR.

FIG. 9 shows that activation of the Wnt pathway and not the NFkB pathway by the GSK3-beta inhibitor Lithium activates latent HIV infected cells. A) S-Lat 46, J-Lat P32, J-Lat A2, J-Lat 11.1, S-Lat 24, S-Lat 9 and S-Lat 30 latently infected Jurkat and SupT1 cell lines were treated with Lithium Chloride (10 mM) for 2 hours, and GFP expression was monitored by FACS analysis to determine activation of the latent LTR. B) J-Lat P32 and (C) S-Lat 30 Jurkats and SupT1 cell lines harbouring latently infected HIV were either untreated or treated with 2, 5 or 10 mM LiCl as indicated. Expression of HIV-LTR driven P24 and GFP, and endogenous Wnt target genes AXIN2, MMP9, TCF1, as well as control GAPDH and NFkB target genes TLR2 and ICAM1 was monitored by qRT-PCR 72 hours after treatment. Expression data are presented as fold induction normalized to Cyclophillin A control. Treatment of Jurkat cells with Lithium (2 mM-10 mM) does not activate the NFkB target genes TLR2 and ICAM1 while treatment with the NFkB activator Prostratin efficiently activates the NFkB target genes (B).

FIG. 10 shows that Lithium and SAHA synergistically activate the latent HIV LTR in HIV-infected cells. A) S-Lat 9, S-Lat 30, and J-Lat T44 harbouring a latently infected HIV-derived LTR-Tat-IRES-GFP virus, and J-Lat 11.1, which contains an integrated full length HIV virus harbouring GFP in place of Nef, were either untreated or treated with 2, 5, 10 mM LiCl, 500 μM SAHA alone or in combination as indicated. GFP expression was monitored by FACS analysis and is expressed as % GFP positive cells. B) S-Lat 30 and J-Lat 11.1 latently infected cell lines were either untreated or treated with LiCl, and SAHA alone or in combination as indicated. Expression of GFP, P24, and the Wnt target genes AXIN2, MMP9, and TCF1 was monitored by qRT-PCR as indicated 72 hours after treatment. Expression data are presented as fold induction normalized to Cyclophillin A control.

FIG. 11 shows that Lithium treatment activates latent HIV in ex-vivo HIV-infected primary CD4+ T cells. A) Lithium treatment activates Wnt and not NFkB target genes in memory CD4+ T cells. Memory CD4+ T cells were untreated or treated with Lithium or prostratin as indicated. mRNA expression of Wnt target genes AXIN2 and TCF1, as well as NFkB target genes TLR2 and ICAM1 was monitored by qRT-PCR 15 hours after treatment. Expression data are presented as fold induction normalized to Cyclophillin A control B) Primary CD4+ T cells were purified from buffy coats from healthy donors and infected ex-vivo with HIV-derived retroviral vector LTR-Tat-IRES-GFP. GFP negative cells comprising uninfected and latently infected cells were sorted by flow cytometry and treated with Lithium (1-5 mM) as indicated. GFP expression was monitored by FACS analysis to determine activation of the latent HIV LTR.

### DETAILED DESCRIPTION OF THE INVENTION

### Definitions

As used herein, the term “retrovirus” refers to a virus having as its genetic material ribonucleic acid (RNA) which is transcribed into DNA which is inserted into the host genome. Examples of retroviruses include HTLV-I, HTLV-II STLV-I, and the lentivirus family including HIV (HIV-1 and HIV-2), visna virus, equine infectious anemia virus, feline immunodeficiency virus and bovine immunodeficiency virus. A retrovirus is duplicated in a host cell using the reverse transcriptase enzyme to produce DNA from its RNA genome. The DNA is then incorporated into the host's genome by an integrase enzyme. The virus thereafter replicates as part of the host cell's DNA. Retroviruses are enveloped viruses that belong to the viral family Retroviridae.

The term “HIV” is used herein to refer to the human immunodeficiency virus. The HIV virus is a hyper-mutable retrovirus, having diverged into two major subtypes (HIV-1 and HIV-2), each of which has many subtypes. However, compounds and methods of the present invention can activate the LTR promoters from all HIV and other retroviruses which are either similar to HIV-1 in the LTR region, or in which b-catenin recruitment to the LTR results in LTR promoter activation. The present disclosure provides a ChIP assay which can be used to determine whether b-catenin recruitment to the LTR to a homologous LTR region from a retrovirus other than HIV-1 occurs. The present disclosure also provides methods to determine if the LTR promoter is activated (see below). Thus, the term “HIV” used herein, unless otherwise indicated, is also used with reference to any retrovirus which is regulated by an LTR promoter or LTR promoter homologue which shows activation of the LTR promoter or LTR promoter homologue by Wnt pathway activators.

The term “LTR” as used herein, refers to the retroviral long terminal repeat, which functions as the retroviral promoter and in particular to the 5′ LTR. These sequences are found in the retroviral genome and in retrotransposons, usually at 3′ and 5′ ends flanking the functional genes. The LTRs are partially transcribed into an RNA intermediate, followed by reverse transcription into complementary DNA (cDNA) and ultimately dsDNA (double-stranded DNA) with full LTRs. The LTRs then mediate integration of the retroviral DNA via an LTR specific integrase into another region of the host chromosome. The HIV genes coding for the viral proteins that form the core of the retroviral virion (gag), the enzymes responsible for reverse transcription (pol) and the envelope glycoproteins (env) as well as other known genes responsible for viral regulation (tat, rev, nef, vif, vpr and vpu) are contained between the LTRs, which are critical for gene transcription of the viral genome. The term “LTR” is used in its broadest meaning, to refer to the structural, promoter, and enhancer elements located in the terminal regions of the RNA or transcribed DNA of a retroviral genome with similar or the same promoter and enhancer elements as HIV-1.

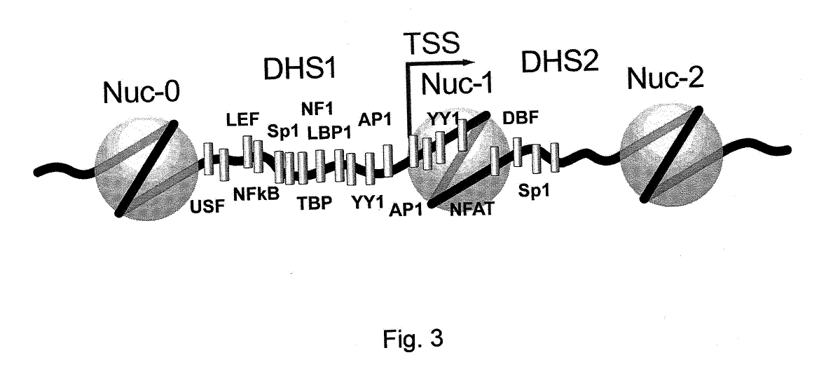

The term “LTR activation”, as used herein refers to the process of transcription of a retroviral LTR when integrated in a host cell genome, in particular to de-repression of LTR transcription in latent host cells. The occurrence of LTR activation may be determined by direct or indirect detection of LTR transcription, such as by detecting LTR transcripts or by detecting LTR transcription of a reporter gene or HIV gene transcription e.g. p24/Gag RT-PCR (Jordan et al., 2001, EMBO J. 20:1726-1738; Jordan et al., 2003, EMBO J. 22:1868-1877; Rouet & Rouzioux, 2007, Clin Lab. 53(3-4):135-48). Alternatively, or in addition, the occurrence of LTR activation may be determined by the disruption of nucleosome nuc-1 positioned immediately downstream of the transcription start site (TSS) (Rafati et al., 2011 PLos Biology 9(11) e1001206; Verdin et al., 2003. EMBO J. 12:3249-3259) (See FIG. 3). Alternatively, or in addition, the occurrence of LTR activation may be determined by observing virus replication in the host cell, detecting an increase in viral load in a host or host cell, and/or detecting retroviral host cell destruction (by reduced host cell count) (Wang S, Xu F, Demirci U. “Advances in developing HIV-1 viral load assays for resource-limited settings”. Biotechnol Adv. 2010; 28(6): 770-781; Gatanaga et al. 2009 Clin Infect Dis. 48(2):260-262). LTR activation may thus be inferred from an increase in the level of LTR transcripts or of a reporter gene as indicated above, to a reduction in nucleosome occupancy of nucleosome nuc-1 in host cell DNA, and/or to an increase in virus replication, viral load or host cell destruction by at least 10%, more preferred at least 20%, more preferred at least 30%, more preferred at least 50%, more preferred at least 70%, more preferred at least 90%, more preferred at least 100%, relative to a level of LTR transcription in the absence of the molecule.

The term “host cell”, as used herein, refers to a mammalian cell, in particular a human cell. Preferred host cells in aspects of this invention are CD4+ T cells, more preferably resting memory CD4+ T cells.

The term “latent host cell”, as used herein, refers to host cells that harbour replication competent virus, blocked at the level of transcription. Latent host cell, or latent cells, are retrovirus infected cells that are not actively producing virus but retain the capacity to do so.

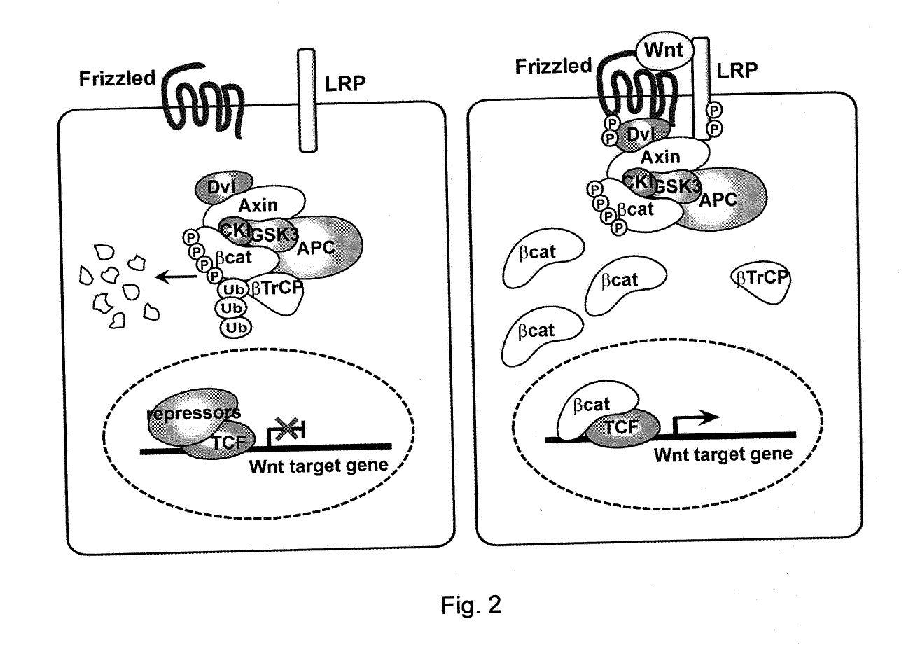

The term “Wnt pathway” or “Wnt signaling pathway”, as used interchangeably herein, refers to the intracellular signaling cascades often referred to as the canonical/beta-catenin pathway, which may be activated through exposure of cells to extracellular Wnt ligands (see FIG. 2). Alternatively, the Wnt signalling pathway may be activated by ligands or agonists of LGR receptors, such as R-Spondin. Wnt proteins (Wnt ligands) bind to a membrane receptor complex comprised of Frizzled (a family of G protein-coupled receptor proteins) and a Lipoprotein Receptor-related Protein (LRP). The formation of this ligand-receptor complex initiates the intracellular signaling cascade referred to as the canonical/beta-catenin pathway. Wnt activation inactivates the function of the “β-catenin destruction complex” (that includes axin, casein kinase 1 (CK1), glycogen synthase kinase-3 (GSK-3), and adenomatous polyposis coli (APC)) that normally promotes the proteolytic degradation of the β-catenin intracellular signaling molecule) as follows: Wnt activation blocks the ubiquitination of phosphorylated b-catenin within the destruction complex. Thus, the destruction complex becomes saturated with phosphorylated b-catenin, which can no longer get ubiquitinylated or proteolytically degraded (Li et al., 2012 Cell, 149(6):1245-56). As a result, the newly synthesized pool of β-catenin stabilizes and accumulates in the cytoplasm, and some β-catenin is able to enter the nucleus and interact with TCF/LEF family of transcription factors to promote specific gene expression. For a complete description of this pathway and its molecules, reference is made to the review of H. Clevers in Cell 127 (2006): 469-480 and Li et al, 2012 Cell), which are incorporated herein by reference in their entirety.

The term “Wnt pathway signaling”, as used herein, refers to activation of the Wnt pathway.

The term “Wnt pathway activator”, or its equivalent “activator of the Wnt pathway”, as used herein, refers to a Wnt ligand or a Wnt pathway agonist. A Wnt agonist is defined as an agent that activates TCF/LEF-mediated transcription in a cell. Wnt agonists are, therefore, selected from true Wnt agonists (or Wnt ligands) that bind and activate a Frizzled receptor family member including any and all of the Wnt family proteins, an inhibitor of intracellular β-catenin degradation, and activators of TCF/LEF. The Wnt agonist stimulates a Wnt activity in a cell by at least 10%, more preferred at least 20%, more preferred at least 30%, more preferred at least 50%, more preferred at least 70%, more preferred at least 90%, more preferred at least 100%, relative to a level of Wnt activity in the absence of the molecule.

As is known to a skilled person, a Wnt activity can be determined by measuring the transcriptional activity of Wnt, for example, by pTOPFLASH and pFOPFLASH TCF luciferase reporter constructs (Korinek et al., 1997, Science 275:1784-1787). Alternative measurements of Wnt activation comprise quantitation/measurement of an increase in levels of mRNA or protein expression of endogenous Wnt target genes such as AXIN2, TCF7, MMP2, MMP9, ZCCHC12, and other known or as yet unknown Wnt-target genes in T cells. Increase in mRNA expression of endogenous Wnt target genes can be determined by quantitative RT-PCR, and by gene expression array analysis (Mahmoudi et al., 2009; Li et al., 2012). Increase in protein expression levels can be determined by Western blot analysis of cell lysates using antibodies specific for the protein product of Wnt target genes or by Mass Spec analysis.

Furthermore, activation of the Wnt pathway may be determined by an increase in levels of de-phospho active betacatenin protein in the cell using an antibody specific for de-phosphorylated active betacatenin (Li et al., 2012). In addition, activation of the Wnt pathway may be determined by an increase in levels of total and S33 phosphorylated beta catenin immunoprecipitating with the endogenous Axin1 beta-catenin destruction complex (Li et al., 2012).

A Wnt agonist comprises a secreted glycoprotein including Wnt-1/Int-1; Wnt-2/Irp (Int-1-related Protein); Wnt-2b/13; Wnt-3/Int-4; Wnt-3a (R&D Systems); Wnt-4; Wnt-5a; Wnt-5b; Wnt-6 (H. Kirikoshi et al., 2001, Biochem. Biophys. Res. Com. 283:798-805); Wnt-7a (R&D Systems); Wnt-7b; Wnt-8a/8d; Wnt-8b; Wnt-9a/14; Wnt-9b/14b/15; Wnt-10a; Wnt-10b/12; Wnt-11; and Wnt-16. An overview of human Wnt proteins is provided in “THE WNT FAMILY OF SECRETED PROTEINS,” R&D Systems Catalog, 2004. Further Wnt agonists include the R-spondin family of secreted proteins, which is implicated in the activation and regulation of the Wnt signaling pathway and which is comprised of four members (R-spondin 1 (NU206, Nuvelo, San Carlos, Calif.), R-spondin 2 ((R&D Systems), R-spondin 3, and R-spondin-4); and Norrin (also called Norrie Disease Protein or NDP) (R&D Systems), which is a secreted regulatory protein that functions like a Wnt protein in that it binds with high affinity to the Frizzled-4 receptor and induces activation of the Wnt signaling pathway (Kestutis Planutis et al. (2007) BMC Cell Biol. 8:12). A small-molecule agonist of the Wnt signaling pathway, an aminopyrimidine derivative, was recently identified and is also expressly included as a Wnt agonist (Liu et at (2005) Angew Chem. Int. Ed. Engl. 44:1987-90). Exemplary Wnt proteins which may be used in the present invention include one or more of Wnt1, Wnt2, Wnt3, Wnt3a, Wnt4, Wnt10, Wnt 14, Wnt14b, Wnt15, and Wnt16, among other Wnt proteins. The use of Wnt3a is preferred.

In a preferred embodiment, the Wnt agonist is selected from one or more of a Wnt family member, R-spondin 1-4, Norrin, and a GSK-inhibitor.

In a further preferred embodiment, the Wnt agonist comprises or consists of R-spondin 1.

In a preferred embodiment, a Wnt agonist is selected from the group consisting of R-spondin, Wnt-3a and Wnt-6.

More preferably, R-spondin and Wnt-3a are both used simultaneously as Wnt agonist. This combination is particularly preferred since this combination surprisingly has a synergetic effect.

The term “GSK inhibitor” is used to describe a compound which inhibits GSK (especially GSK3, including GSK3a or GSK3β). GSK (glycogen synthase kinase) preferably means GSK3β. Known GSK-inhibitors comprise small-interfering RNAs, lithium (in any form but preferably as LiCl and/or lithium carbonate), kenpaullone, 6-Bromoindirubin-30-acetoxime, SB 216763 and SB 415286, CHIR-99021, and FRAT-family members and FRAT-derived peptides that prevent interaction of GSK-3 with axin. An overview is provided by Meijer et al., (2004) Trends in Pharmacological Sciences 25:471-480, which is hereby incorporated in its entirety by reference. Methods and assays for determining a level of GSK-3 inhibition are known to a skilled person and comprise, for example, the methods and assay as described in Liao et al. 2004, Endocrinology 145(6):2941-9).

Examples of preferred GSK inhibitors for use in the present invention include one or more of the following: BIO (2′Z,3′E)-6-Bromoindirubin-3′-oxime (GSK3 Inhibitor IX); BIO-Acetoxime (2′Z,3′E)-6-Bromoindirubin-3′-acetoxime (GSK3 Inhibitor X); (5-Methyl-1H-pyrazol-3-yl)-(2-phenylquinazolin-4-yl)amine (GSK3-Inhibitor XIII); Pyridocarbazole-cyclopenadienylruthenium complex (GSK3β Inhibitor XV); TDZD-8 4-Benzyl-2-methyl-1,2,4-thiadiazolidine-3,5-dione (GSK3β Inhibitor I); 2-Thio(3-iodobenzyl)-5-(1-pyridyl)[1,3,4]-oxadiazole (GSK3β Inhibitor II); OTDZT 2,4-Dibenzyl-5-oxothiadiazolidine-3-thione (GSK3β Inhibitor III); α-4-Dibromoacetophenone (GSK3β Inhibitor VII); AR-A014418 N-(4-Methoxybenzyl)-N′-(5-nitro-1,3-thiazol-2-yl)urea (GSK-3β Inhibitor VIII); 3-(1-(3-Hydroxypropyl)-1H-pyrrolo[2,3-b]pyridin-3-yl]-4-pyrazin-2-y-l-pyrrole-2,5-dione (GSK-3β Inhibitor XI); TWS119 pyrrolopyrimidine compound (GSK38 Inhibitor XII); L803 H-KEAPPAPPQSpP-NH2 or its Myristoylated form (GSK3β Inhibitor XIII); 2-Chloro-1-(4,5-dibromo-thiophen-2-yl)-ethanone (GSK3β Inhibitor VI), and CHIR99021, an aminopyrimidine.

Preferred GSK inhibitors for use in the present invention include, BIO (GSK-3 IX), CHIR-99021, and lithium.

As used herein, the term “histone deacetylases (HDAC)” refers to enzymes capable of removing the acetyl group bound to the lysine residues in the N-terminal portion of histones or in other proteins. Generally, HDACs can be subdivided into four classes, on the basis of structural homologies. Class I HDACs (HDAC 1, 2, 3 and 8) are similar to the RPD3 yeast protein and are located in the cell nucleus. Class II HDACs (HDAC 4, 5, 6, 7, 9 and 10) are similar to the HDA1 yeast protein and are located both in the nucleus and in the cytoplasm. Class III HDACs are a structurally distinct form of NAD-dependent enzymes correlated with the SIR2 yeast protein. Class IV (HDAC 11) consists at the moment of a single enzyme having particular structural characteristics. The HDACs of classes I, II and IV are zinc enzymes and can be inhibited by various classes of molecule: hydroxamic acid derivatives, cyclic tetrapeptides, short-chain fatty acids, aminobenzamides, derivatives of electrophilic ketones, and the like. Class III HDACs are not inhibited by hydroxamic acids, and their inhibitors have structural characteristics different from those of the other classes. The HIV LTR is repressed by class I and II HDACs. VPA, TSA, etc, these are class I and II HDAC inhibitors.

As used herein, the “histone deacetylase inhibitor”, abbreviated herein as HDACi, in relation to the present invention is to be understood as meaning any molecule of natural, recombinant or synthetic origin capable of inhibiting the activity of at least one of the enzymes classified as histone deacetylases, preferably of class I, class II or class IV.

Suitable HDACi's include, but are not limited to hydroxamic acid derivatives, including trichostatin A (TSA), vorinostat (suberoylanilide hydroxamic acid, SAHA), belinostat (PXD101), panobinostat (LBH589), LAQ824, PCI-24781, SB939, givinostat (ITF2357) dacinostat (LAQ824), resminostat (45C-201), resminostat (4SC-202), pyroxamide, JNJ-24681585, CHR-2845, CHR-3996, CUDC-101, azelaic-1-hydroxamate-9-anilide (AAHA), CRA-024781, bombesin-2 (BB2) receptor antagonist, JNJ-16241199, Oxamflatin, CG-1521, CG-1255, SK-7068, SK-7041, m-carboxycinnamic acid bis-hydroxamide (CBHA), Scriptaid (N-Hydroxy-1,3-dioxo-1H-benz[de]isoquinoline-2(3H)-hexan amide), SB-623, SB-639, SB-624, Panobinostat (LBH-589), NVP-LAQ824, CG-200745; cyclic tetrapeptides/epoxides and depsipeptides, including Apicidine, Trapoxin-A, Trapoxin-B, cyclic hydroxamic acid-containing peptide 1 (CHAP-1), CHAP-31, CHAP-15, chlamidocin, HC-Toxin, WF-27082B, Romidepsin (FR901228, FK228), Spiruchostatin A, Depudesin, Triacetylshikimic acid, Cyclostellettamine FFF1, Cyclostellettamine FFF2, Cyclostellettamine FFF3, Cyclostellettamine FFF4; benzamides, including entinostat (MS-275), tacedinaline (CI-994), and mocetinostat (MGCD0103), ITF-2357, N-hydroxy-4-(3-methyl-2-phenyl-butyrylamino)benzamide (HDAC-42), MGCD-0103, PX-117794, Belinostat, sulfonamide hydroxamic acid; isothiocyanates, including sulforaphane; isoflavonoids, including Pomiferin; electrophilic ketones, including a thfluoromethyl ketone or an alpha-keto amide; aliphatic acid derivatives or short-chain fatty acids including phenylbutyrate, tributyrin, hyaluronic acid butyric acid ester, pivaloyloxymethyl butyrate (AN-9), and valproic acid (VPA); pharmaceutically acceptable salts thereof, and combinations thereof. A highly preferred HDACi used in aspects of the present invention is VPA, and/or SAHA.

Many other HDAC inhibitors may be used in aspects of the present invention, many of which are for instance disclosed in WO2011/006040, the disclosure of which is incorporated herein by reference in its entirety.

In the context of this specification, “pharmaceutically acceptable salts” include, but are not limited to, those formed from: acetic, ascorbic, aspartic, benzoic, benzenesulfonic, citric, cinnamic, ethanesulfonic, fumaric, glutamic, glutaric, gluconic, hydrochloric, hydrobromic, lactic, maleic, malic, methanesulfonic, naphthoic, hydroxynaphthoic, naphthalenesulfonic, naphthalenedisulfonic, naphthaleneacrylic, oleic, oxalic, oxaloacetic, phosphoric, pyruvic, p-toluenesulfonic, tartaric, trifluoroacetic, triphenylacetic, tricarballylic, salicylic, sulfuric, sufamic, sulfanilic and succinic acid.

In the context of this specification, the terms “treatment” and “treating” refer to any and all uses which remedy a condition or disease or symptoms thereof, prevent the establishment of a condition or disease or symptoms thereof, or otherwise prevent or hinder or reverse the progression of a condition or disease or other undesirable symptoms in any way whatsoever.

In the context of this specification, the term “therapeutically effective amount” includes within its meaning a non-toxic amount of a compound sufficient to provide the desired therapeutic effect. The exact amount will vary from subject to subject depending on the age of the subject, their general health, the severity of the disorder being treated and the mode of administration. In addition, it will depend on the substance that is administered. It is therefore not possible to specify an exact “therapeutically effective amount”, however one skilled in the art would be capable of determining a “therapeutically effective amount” by routine trial and experimentation.

The term “antiretroviral therapy” or “ART”, refers to any and all therapies aimed to suppress retrovirus load in a subject, to reduce retrovirus replication in a subject and/or to reduce retrovirus-associated morbidity and mortality. This is usually accomplished by administering to infected subjects a therapeutically effective amount of a combination of antiretroviral drugs, such as CCR5 receptor antagonists, nucleoside and nucleotide reverse transcriptase inhibitors (NRTI and NtRTI, respectively), non-nucleoside reverse transcriptase inhibitors (NNRTI), protease inhibitors (PIs) and integrase inhibitors. Antiretroviral therapy includes reference to such treatments as combination antiretroviral therapy (cART) and highly active antiretroviral therapy (HAART). Both treatments target the virus after transcription has been activated (i.e. treatment relying on active virus). HAART treatment includes reference to intensified HAART and normal HAART. Guidelines for antiretroviral therapy of HIV are for instance provided in the World Health Organization publication “Antiretroviral therapy for HIV infection in adults and adolescents: recommendations for a public health approach”—2010 rev., ISBN 978 92 4 159976 4, World Health Organization 2010.

### PREFERRED EMBODIMENTS

The present inventor found that LiCl activates the HIV LTR in several cell line model systems and de-represses latently infected HIV in Jurkat latent (J-Lat) cell lines containing latent integrated single infectious cyle full length HIV or HIV-derived viruses. Without wishing to be bound to theory, it is believed that the effect of LiCl is brought about by inhibiting the enzyme glycogen synthase kinase 3 beta (GSK3b), an essential component of the beta-catenin destruction complex and a component of the Wnt pathway.

This was supported by the finding that treatment of latently infected cells with 6-bromoindirubin-3′-oxime (BIO), another GSK3-specific inhibitor, also activates the LTR in latently HIV infected cells.

More importantly, it was found through chromatin immunoprecipitation (ChIP) experiments that treatment of latent cells with BIO leads to physical recruitment of the beta-catenin protein and enrichment of LEF1 at the HIV LTR, and that LiCl treatment also induced the recruitment of beta-catenin and enrichment of LEF1, to the LTR of latent cells. Further is was found by using ChIP assays that treatment of latent cells with R-spondin/Wnt (a combination of Wnt agonists) induced recruitment of b-catenin and enrichment of LEF1 to the LTR. LEF1 and b-catenin are the molecular effectors of Wnt signaling in T cells. The direct involvement of Wnt signaling in LTR activation in latent cells was confirmed by exposing the latent cells to the Wnt3A ligand and R-spondin as this resulted in synergistic de-repression of HIV latency. WNT and Rspondin were also shown to synergize in activating LTR transcription in latently infected CD4+ T cells. Thus, Wnt pathway activation by natural ligands, and/or functional inactivation of the beta-catenin destruction complex by inhibitors and chemical compounds, which induce beta-catenin nuclear localization, activate the LTR in latent HIV infected cells.

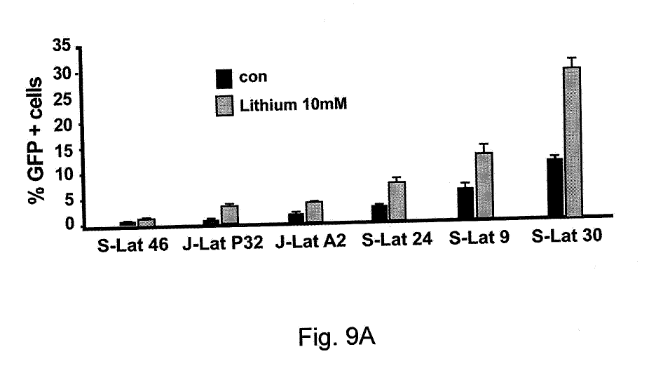

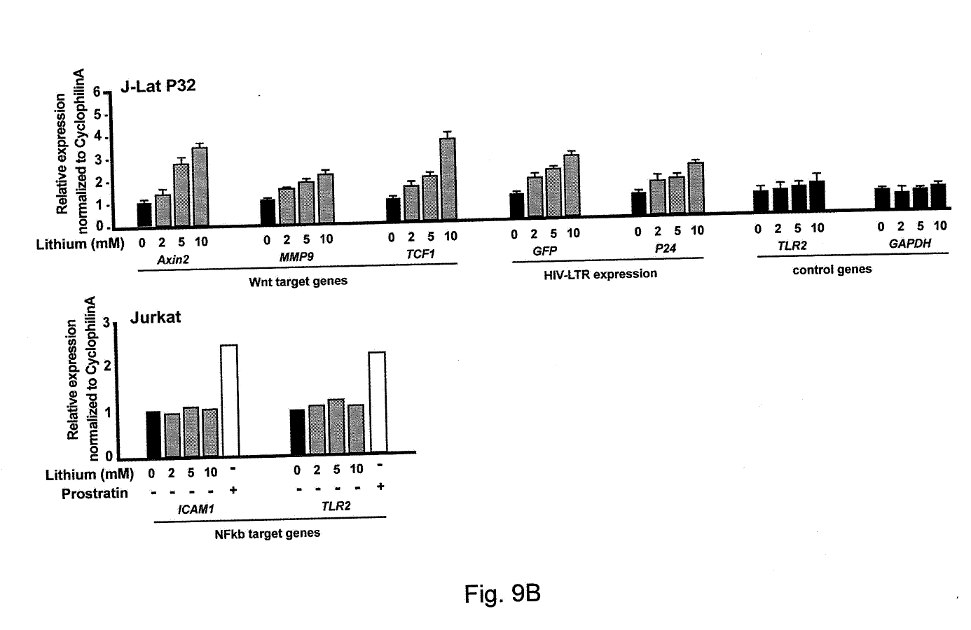

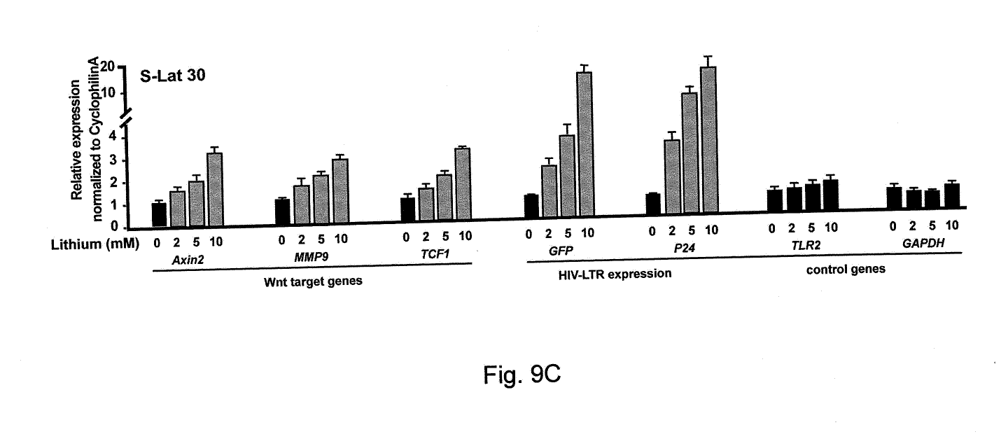

WO2007/121429 discloses a method to eliminate latent HIV reservoirs and uses amongst others prostratin and HDAC inhbitors such as TSA. It furthermore discloses that inducers of NF-κB pathway may be used as activator of latent HIV expression. Lithium is mentioned as NF-κB inducer. The present inventors found that the amount of Lithium to induce NF-κB is much higher than the amount to induce Wnt pathway, see FIG. 9.

Some studies in the prior art show that induction of Wnt represses retroviral replication, however, the present inventors have successfully shown that Wnt activation of latent HIV activates translation of HIV LTR. It is shown that efficient transcriptional activation of HIV p24(Gag) mRNA as well as translation of latent HIV LTR GFP reporter in Wnt-activated latent HIV infected cells takes place upon stimulation of Wnt. In other words, Wnt activation of latent HIV-1, results in expression of HIV mRNA followed by efficient translation of its proteins. Thus, expression of the HIV genome (which is critical for subsequent antigen presentation and initiation/eliciting a cytotoxic T cell response) is activated in response to Wnt activation.

The present inventor further found that LiCl synergizes with Valproic acid (VPA), which is a class I/II histone deacetylase inhibitor, to activate the HIV LTR in 1G5, TZMB1, and J-Lat cells. In fact, it was found that simultaneous treatment of a polyclonal population of cells (containing latent HIV infected cells) with both LiCl and VPA result in synergistic activation or purging of the latently infected cell population.

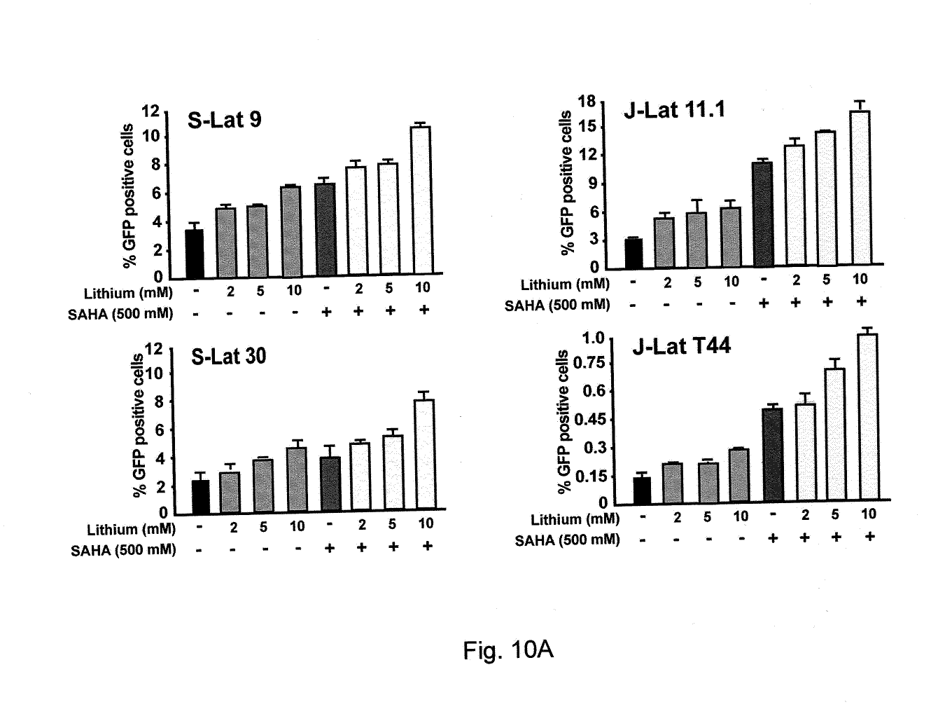

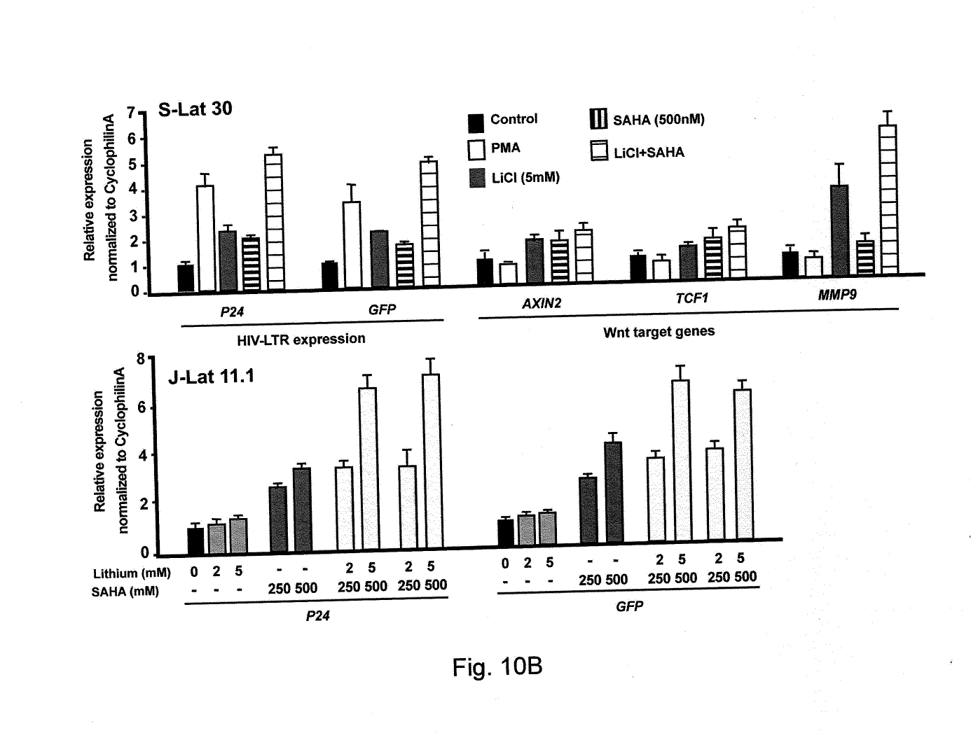

The histone deacetylase inhibitor VPA was previously identified as an activator of latent HIV and investigated in clinical studies for potential to purge the latent HIV infected reservoir in patients (Lehrman et al., 2005 Lancet 366(9485):549-55). 11-7 has also been shown to activate T cells and latent HIV (Nunnari & Pomerantz 2005 Expert Opin Biol Ther. 5(11):1421-6). Prostratin (an inhibitor of PKC signaling; Biancotto et al., 2004 J Virol. 78(19):10507-15) and HMBA (Hexamethylbisacetamide; Choudhary 2008 J Infect Dis. 15; 197(8):1162-70) which interferes with HEXIM-mediated sequestration of CyclinT, necessary for activation of HIV are also useful active compounds for use in the present invention. The present inventor further found that LiCl synergizes with vorinostat (SAHA), see FIG. 10. Hence, it is foreseen in aspects of the present invention that also VPA, 11-7, Prostratin, HMBA, SAHA, and CyclinT are used in combination with Wnt pathway activators and optionally further in combination with HDAC inhibitors, to activate retroviral LTR in latent cells.

Latent HIV infected resting memory CD4+ T cells harbour replication competent virus, blocked at the level of transcription. Transcription of the HIV-1 virus is driven by the LTR and is restricted in vivo. Regardless of the position of virus integration in the host genome, within the 5′LTR, the nucleosomes are strictly deposited at specific positions (Verdin et al., 2003. EMBO J. 12:3249-3259; Rafati et al., 2011 PLos Biology 9(11) e1001206). Chromatin organization of the HIV-1 provirus demonstrate the presence of at least three precisely positioned nucleosomes, nuc-0, nuc-1 and nuc-2 and their intervening nucleosome-free regions (FIG. 3). In particular nuc-1, the nucleosome positioned immediately downstream of the transcription start site (TSS) is repressive to transcription and surrounded by two large domains of nucleosome-free DNA. Following activation, nuc-1 becomes rapidly and specifically disrupted.

In the immediate-early phase of HIV infection, cellular transcription factors activate transcription from the viral promoter in the 5′-LTR, leading to accumulation of viral Tat protein, a potent transactivator, causing a Tat-dependent positive feedback loop and surge in transcription. Tat also leads to the remodelling of nuc-1, the nucleosome positioned immediately downstream of TSS. It has been reported that Tat recruits many co-activators including the SWI/SNF chromatin-remodelling complex to activate LTR transcription.

In addition to remodelers, many signaling pathways, cellular cofactors have been shown to regulate HIV LTR activity and control post-integration latency. Thus, many known molecular components and putative, as yet unknown LTR regulators may be subject to small molecule inhibition, resulting in de-repression of latent HIV.

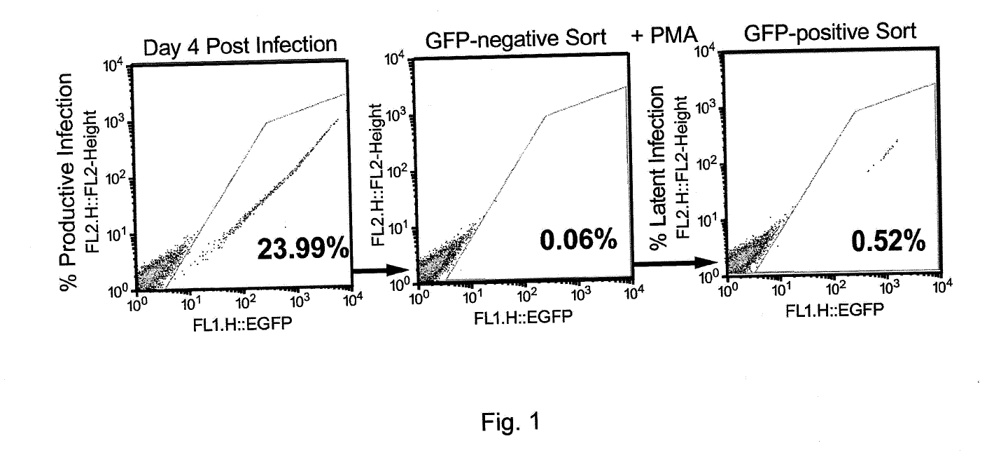

Research suggests that latent HIV infected cells contain replication-competent integrated HIV-1 genomes blocked at the transcriptional level. Using a strategy similar to that described previously (Jordan et al., 2003, EMBO J. 22:1868-1877), the generation of several latent HIV infected clonal and polyclonal SupT1 and Jurkat cells lines was accomplished. Briefly, CD4+ Jurkat or SupT1 T cells were infected at low multiplicity of infection with an HIV-1 derived virus containing a GFP reporter, LTR-GFP, LTR-Tat-IRES-GFP or a full-length HIV-1 genome expressing GFP in place of Nef and a frame shift mutation in env. Using Flow Cytometry (FACS) the GFP negative population, containing uninfected as well as presumably latently infected cells was sorted. Treatment of the GFP negative population with PMA or other LTR activators led to activation and GFP expression of the latently infected population, which were FACS sorted and expanded as clonal or polyclonal populations that are latent (GFP negative) but can be activated to express GFP. The Jurkat Latent (J-Lat)/SupT1 Latent (S-Lat) model system reflecting the transcriptional state of HIV latency has opened new doors to understanding the HIV life cycle and provides a powerful tool to distinguish between active and latent HIV infected cells. The above method for generation of the CD4+ T cell line model system reflecting HIV latency is depicted in FIG. 1.

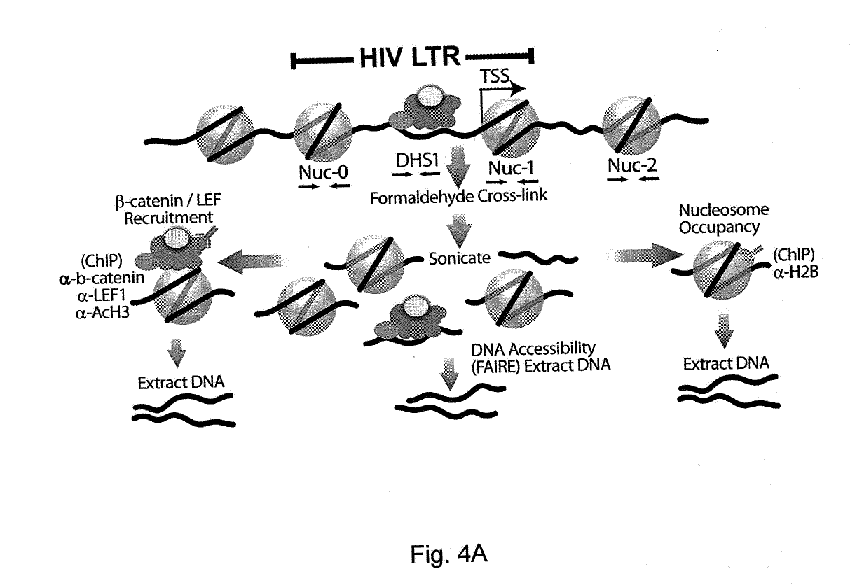

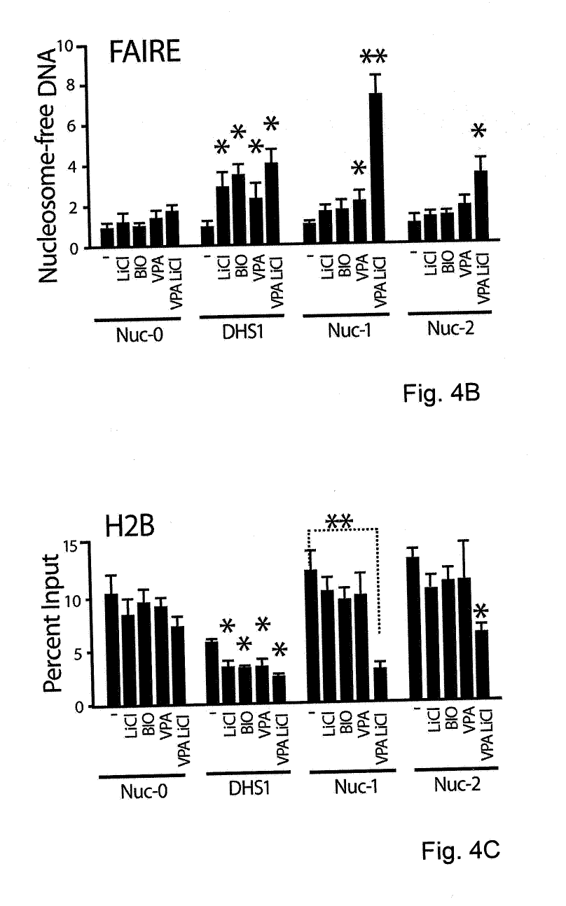

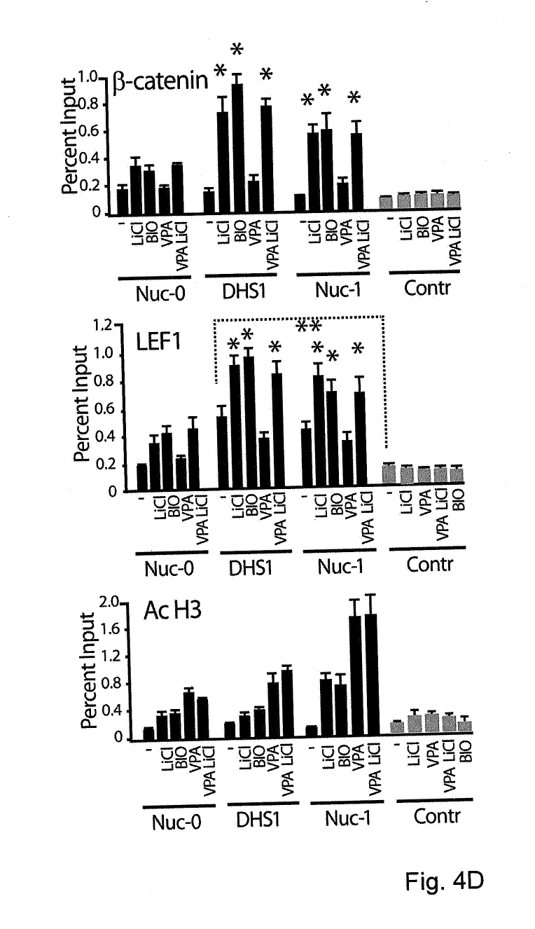

Using the J-Lat/S-Lat cell system, the inventor previously developed a combinatorial approach based on formaldehyde crosslinking to simultaneously determine nucleosome density, DNA accessibility and cofactor recruitment to distinct regions at the HIV LTR in latent versus activated states (FIG. 4A).

It is an aspect of the present invention that re-activation of latent HIV in CD4+ T cells is brought about by Wnt pathway-mediated activation of the LTR wherein activation of the Wnt pathway is brought about by natural ligands or chemical molecules or inhibitors. It should be noted that chemical Wnt activators as well as biological wnt activators are shown to activate latent HIV transcription. In the examples, the chemical compounds tested and shown to be effective such as lithium salts, and small molecules such as BIO and CHIR-99021, are chemically very different. In addition, the biomolecules R-spondin, Wnt3A, and constitutively active β-catenin, are both protein, but differ greatly as R-spondin is a 27 kDa protein, and b-catenin is a 88 kDa protein. It is also shown that these biomolecules are effective in activating latent HIV. These results from the examples show that irrespective of the nature of compound that activates the Wnt pathway, as long as the Wnt pathway is activated, it will activate latent HIV transcriprion and translation.

The proposed strategy is very beneficially used in combination with other de-repressor of the HIV LTR, such as valproic acid (VPA) and or vorinostat (SAHA), which are histone deacetylase inhibitors. Such re-activation of latent HIV in CD4+ T cells will deplete the reservoir of latently infected cells. In fact a synergistic effect between Wnt pathway activators and VPA and/or SAHA in activating HIV latency was found, which may be extended to other histone deacetylase inhibitors.

Broadly, the present invention is drawn to methods for modelling transcriptional regulation of the HIV LTR, using multiple pathways involved in the regulation of HIV latency and proposes combinatorial treatment for activation, as a combinatorial approach has shown to be more effective and synergistically purges the reservoir of latent cells.

The present invention provides a method for increasing retrovirus transcription in an infected eukaryotic cell. The present inventor found that activators of the Wnt pathway, such as β catening and/or the ligands Wnt3A and R-spondin, or the GSK3 inhibitors BIO, CHIR-99021 and/or lithium, activate the HIV LTR promoter function in latent HIV infected CD4+ T cells. It was thus inferred that Wnt pathway activation can thus be used to purge HIV from latently infected cells.

In general, the method not only relates to latently infected cell, but also to cells having low level of transcription. Hence, productively HIV infected cells with low level or partial LTR activity can also favourably be subjected to a method of the present invention.

In latently infected cells, the production of virus particles is arrested due to repression of the transcription of the retrovirus genomic insert. A method of the invention aims to activate the repressed retrovirus.

In accordance with methods as described herein, de-repression of transcription will occur by increasing Wnt pathway signaling in said cell such that the retroviral long terminal repeat (LTR) is activated or de-repressed.

The skilled person is aware of methods for increasing Wnt pathway signaling in cells. Wnt pathway signaling in the context of this invention is to be understood as resulting in a physiologically relevant level of beta-catenin in the cell as a result of beta-catenin stabilization (or lack of destruction). A physiologically relevant level of beta-catenin is a level that results in recruitment of beta-catenin at the LTR. In particular as described herein.

For example, activation of the Wnt signaling pathway may be measured/quantitated by an increase In TCF-driven transcription in the cell after introduction of TOPFLASH (plasmid with tandem TCF/LEF consensus sites driving expression of the luciferase gene) into the cell via transfection/nucleofection. Increase in TOPFLASH (TCF-driven) and not FOPFLASH (TCF-mutant reporter) indicates activation of the Wnt signaling pathway.

Another way of measuring activation of the Wnt signaling pathway may be by quantitating/measuring an increase in levels of mRNA or protein expression of endogenous Wnt target genes such as AXIN2, TCF7, MMP2, MMP9, ZCCHC12, and other known or as yet unknown Wnt-target genes in T cells. Increase in mRNA expression of endogenous Wnt target genes can be determined by quantitative RT-PCR, and by gene expression array analysis (Mahmoudi et al., 2009; Li et al., 2012). Increase in protein expression levels can be determined by Western blot analysis of cell lysates using antibodies specific for the protein product of Wnt target genes or by Mass Spec analysis.

In addition, the activation of the Wnt pathway may be determined by an increase in levels of de-phospho active betacatenin protein in the cell using an antibody specific for de-phosphorylated active betacatenin (Li et al., 2012).

Furthermore, activation of the Wnt pathway may be determined by an increase in levels of total and S33 phosphorylated beta catenin immunoprecipitating with the endogenous Axin1 beta-catenin destruction complex as we have shown (Li et al., 2012).

The active compounds as described herein (that is the Wnt pathway activators, but also the various other activators and inhibitors as described above) are useful as ligands, activators or inhibitors in the methods of as described herein. The active compounds as described herein may suitably be administered to a cell in the form of pharmaceutical compositions as described herein below for human subjects.

The present invention in one preferred embodiment relates to the administration of the preferred Wnt pathway activator lithium, alone, to a cell or subject in a method for depleting the latent reservoir in HIV infected cells or subjects, preferably antiretroviral therapy (ART)-treated subjects. Co-treatment of lithium and VPA and/or SAHA together is highly preferred in methods of the present invention as these compounds synergistically activate the HIV LTR and activate/deplete/purge latent HIV infections. In a combination with ART, this provides for a curative therapy for HIV infections because the elimination of the latently HIV infected reservoir of memory T cells in combination with modern ART regimens can potentially eradicate HIV from infected patients. Antiretroviral therapy need not be described in detail herein. Reference is made to Scourfield A, Waters L, Nelson M. (2011) Expert Rev Anti Infect Ther. (11):1001-11.

The invention further relates to a method for the treatment or prophylaxis of a condition associated with latent retrovirus infection in a subject in need thereof comprising administration to the subject of a therapeutically effective amount of the active compounds as described herein, as defined above. The condition associated with latent retrovirus infection may be HIV infection.

The invention also relates to a method for the treatment or prophylaxis of a condition associated with latent retrovirus infection in a subject in need thereof comprising administration to the subject of a therapeutically effective amount of compounds that activate Wnt pathway.

Furthermore, the invention relates to compounds that activate Wnt pathway for use in the treatment of retrovirus infection. In a preferred embodiment of the invention and/or embodiments thereof, the compounds that activate Wnt pathway are administered in a therapeutically active amount. In a preferred embodiment of the present invention and/or embodiments thereof the amount of lithium does not activate NF-κB. In a preferred embodiment of the present invention and/or embodiments thereof, the amount of lithium is administered in a dose that provides therapeutically active plasma concentration of 0.1-10 mmol/L.

The active compounds as described herein are useful as therapeutic agents in the methods of treatment as described herein. The active compounds as described herein may suitably be administered to a subject (for example a human) in the form of pharmaceutical compositions.

Pharmaceutical compositions include those suitable for oral, parenteral (including subcutaneous, intradermal, intramuscular, intravenous and intraarticular), inhalation (including use of metered dose pressurised aerosols, nebulisers or insufflators), rectal and topical (including dermal, buccal, sublingual and intraocular) administration.

The compositions may conveniently be presented in unit dosage form and may be prepared by any of the methods well known in the art of pharmacy. All methods include the step of bringing the active compounds as described herein into association with a carrier which constitutes one or more accessory ingredients. In general, the compositions are prepared by uniformly and intimately bringing into association the active compounds as described herein with a liquid carrier or finely divided solid carrier, or both and then, if necessary, shaping the product into the desired composition.

Generally, an effective dosage of the active compounds as described herein present in pharmaceutical and other compositions of the present invention is expected to be in the range of about 0.0001 mg to about 1000 mg per kg body weight per 24 hours; about 0.001 mg to about 750 mg per kg body weight per 24 hours; about 0.01 mg to about 500 mg per kg body weight per 24 hours; about 0.1 mg to about 500 mg per kg body weight per 24 hours; about 0.1 mg to about 250 mg per kg body weight per 24 hours, or about 1.0 mg to about 250 mg per kg body weight per 24 hours. More typically, an effective dose range is expected to be in the range of about 1.0 mg to about 200 mg per kg body weight per 24 hours; about 1.0 mg to about 100 mg per kg body weight per 24 hours; about 1.0 mg to about 50 mg per kg body weight per 24 hours; about 1.0 mg to about 25 mg per kg body weight per 24 hours; about 5.0 mg to about 50 mg per kg body weight per 24 hours; about 5.0 mg to about 20 mg per kg body weight per 24 hours, or about 5.0 mg to about 15 mg per kg body weight per 24 hours.

Alternatively, an effective dosage may be up to about 500 mg/m2. Generally, an effective dosage is expected to be in the range of about 25 to about 500 mg/m2, about 25 to about 350 mg/m2, about 25 to about 300 mg/m2, about 25 to about 250 mg/m2, about 50 to about 250 mg/m2, or about 75 to about 150 mg/m2.

Suitable therapeutic range for lithium may be 1 mmol/L of plasma in a subject.

Treatment schemes for HIV infected subjects may comprise for instance treatment with Lithium alone or together with VPA and/or SAHA in order to deplete the latent HIV infected reservoir in accordance with the present invention. Subject may simultaneously receive treatment with the a regimen of stable c-ART (NNRT+, tenofovir +3TC). Very suitably, VPA is administered orally to subjects in a dose of about 50-2500 mg, preferably 500-750 mg, preferably twice daily. The dosage regime may be adjusted to maintain plasma concentrations of 10-500 mg/L, more preferably plasma concentrations of VPA of about 50-100 mg/L.

In a preferred embodiment of the present invention and/or embodiments thereof SAHA is administered in a single dose of about 100-800 mg, more preferably between 200-600 mg, more preferably between 250-500 mg, more preferably, between 300-400 mg. This dose may be repeated after a period such as after 1 week, after two weeks, after 4 weeks, after 5 weeks or even after 6 weeks. The dose may be repeated more than 1 time. Preferably, it is repeated as long as latent retrovirus is present. In a preferred embodiment of the present invention and/or embodiments thereof the plasma level of SAHA is monitored (see e.g Archin et al, Nature (2012) Volume: 487, Pages: 482-485). In a preferred embodiment of the present invention and/or embodiments thereof the plasma concentration of SAHA in a patient is between 25 and 600 ng/mL, more preferably between 50 and 500 ng/mL, more preferably between 100 and 400 ng/mL, more preferably between 150 and 350 ng/ml, more preferably between 200 and 300 ng/ml. The plasma concentration of SAHA is preferably the peak concentration, which is usually achieved 2-8 hours after administration.

Lithium administration may for instance occur in the form of lithium carbonate. A suitable administration scheme is orally. In a preferred embodiment, lithium administration is twice daily. In a preferred embodiment, lithium administration is with an amount of 1-100, more preferably 5-25, still more preferably 10-15 mg of lithium carbonate/kg body weight. Very suitable target therapeutic plasma concentrations are in the range of 0.1-10 mmol/L, more preferably 0.5-5 mmol/L, more preferably 1.0-4.0 mmol/L, more preferably 1.5-3.0 mmol/L, more preferably from 2.0-2.5 mmol/L, in the plasma of the subject. The therapeutic concentrations are preferably monitored. In a preferred embodiment, the therapeutic plasma concentration is below 4.0 mmol/L, more preferably below 1.5 mmol/L as higher concentrations may have side effects such as tremor, ataxia, dysarthria, nystagmus, renal impairment, confusion, and convulsions.

It should be noted that the concentration of lithium needed to induce NF-κB pathway (20-100 mM) are in the range that is much higher than that of induction of the Wnt pathway 0.1-10 mM) FIG. 9 shows that treatment of Jurkat cells with Lithium in a concentration of 2 mmol/L-10 mmol/L does not activate the NFkB target genes.

Treatment periods for treatments as proposed herein may suitably continue for several weeks, such as 4-100 weeks, preferably about 20 weeks.

Preferably, subjects will continue receiving ART treatment during the treatment period.

Compositions suitable for buccal (sublingual) administration include lozenges comprising the active compounds as described herein in a flavoured base, usually sucrose and acacia or tragacanth; and pastilles comprising the active compounds as described herein in an inert base such as gelatine and glycerin or sucrose and acacia.

Compositions comprising the active compounds as described herein suitable for oral administration may be presented as discrete units such as gelatine or HPMC capsules, cachets or tablets, each containing a predetermined amount of the active compounds as described herein, as a powder, granules, as a solution or a suspension in an aqueous liquid or a non-aqueous liquid, or as an oil-in-water liquid emulsion or a water-in-oil liquid emulsion. The active compounds as described herein may also be present in a paste.

When the compositions comprising the active compounds as described herein are formulated as capsules, the active compounds may be formulated with one or more pharmaceutically acceptable carriers such as starch, lactose, microcrystalline cellulose, silicon dioxide and/or a cyclic oligosaccaride such as cyclodextrin. Additional ingredients may include lubricants such as magnesium stearate and/or calcium stearate. Suitable cyclodextrins include α-cyclodextrin, β-cyclodextrin, □□-cyclodextrin, 2-hydroxyethyl-β-cyclodextrin, 2-hydroxypropyl-cyclodextrin, 3-hydroxypropyl-β-cyclodextrin and tri-methyl-β-cyclodextrin. The cyclodextrin may be hydroxypropyl-β-cyclodextrin. Suitable derivatives of cyclodextrins include Captisol® a sulfobutyl ether derivative of cyclodextrin and analogues thereof as described in U.S. Pat. No. 5,134,127.

Tablets may be prepared by compression or moulding, optionally with one or more accessory ingredients. Compressed tablets may be prepared by compressing in a suitable machine the active compounds as described herein in a free-flowing form such as a powder or granules, optionally mixed with a binder, lubricant (for example magnesium stearate or calcium stearate), inert diluent or a surface active/dispersing agent. Moulded tablets may be made by moulding a mixture of the powdered active compounds as described herein moistened with an inert liquid diluent, in a suitable machine. The tablets may optionally be coated, for example, with an enteric coating and may be formulated so as to provide slow or controlled release of the active compounds as described herein therein.

Compositions for parenteral administration include aqueous and non-aqueous sterile injectable solutions which may contain anti-oxidants, buffers, bacteriostats and solutes which render the formulation isotonic with the blood of the intended recipient, and which may include suspending agents and thickening agents. A parenteral composition may comprise a cyclic oligosaccaride such as hydroxypropyl-β-cyclodextrin. The compositions may be presented in unit-dose or multi-dose containers, for example sealed ampoules and vials, and may be stored in a freeze-dried (lyophilised) condition requiring only the addition of the sterile liquid carrier, for example saline or water-for-injection, immediately prior to use.

Compositions suitable for transdermal administration may be presented as discrete patches adapted to remain in intimate contact with the epidermis of the recipient for a prolonged period of time. Such patches suitably comprise the active compounds as described herein as an optionally buffered aqueous solution of, for example, 0.1 M to 0.2 M concentration with respect to the compound.

Compositions suitable for transdermal administration may also be delivered by iontophoresis, and typically take the form of an optionally buffered aqueous solution of the active compound. Suitable compositions may comprise citrate or Bis/Tris buffer (pH 6) or ethanol/water and contain from 0.1 M to 0.2 M of the active compounds as described herein.

Spray compositions for topical delivery to the lung by inhalation may, for example be formulated as aqueous solutions or suspensions or as aerosols, suspensions or solutions delivered from pressurised packs, such as a metered dose inhaler, with the use of a suitable liquefied propellant. Suitable propellants include a fluorocarbon or a hydrogen-containing chlorofluorocarbon or mixtures thereof, particularly hydrofluoroalkanes, e.g. dichlorodifluoromethane, trichlorofluoromethane, dichlorotetrafluoroethane, especially 1,1,1,2-tetrafluoroethane, 1,1,2,2,3,3,3-heptafluoro-n-propane or a mixture thereof. Carbon dioxide or other suitable gas may also be used as propellant. The aerosol composition may be excipient free or may optionally contain additional composition excipients well known in the art, such as surfactants e.g. oleic acid or lecithin and cosolvents e.g. ethanol. Pressurised compositions will generally be retained in a canister (e.g. an aluminium canister) closed with a valve (e.g. a metering valve) and fitted into an actuator provided with a mouthpiece.

Medicaments for administration by inhalation desirably have a controlled particle size. The optimum particle size for inhalation into the bronchial system is usually 1-10 μm, preferably 2-5 μm. Particles having a size above 20 μm are generally too large when inhaled to reach the small airways. When the excipient is lactose it will typically be present as milled lactose, wherein not more than 85% of lactose particles will have a MMD of 60-90 μm and not less than 15% will have a MMD of less than 15 μm.

Compositions for rectal administration may be presented as a suppository with carriers such as cocoa butter or polyethylene glycol, or as an enema wherein the carrier is an isotonic liquid such as saline. Additional components of the compositions may include a cyclic oligosaccaride, for example, a cyclodextrin, as described above, such as hydroxypropyl-□-cyclodextrin, one or more surfactants, buffer salts or acid or alkali to adjust the pH, isotonicity adjusting agents and/or anti-oxidants.

Compositions suitable for topical administration to the skin preferably take the form of an ointment, cream, lotion, paste, gel, spray, aerosol, or oil. Carriers which may be used include Vasoline, lanoline, polyethylene glycols, alcohols, and combination of two or more thereof. The active compounds as described herein is generally present at a concentration of from 0.1% to 20% w/w, or from 0.5% to 5% w/w. Examples of such compositions include cosmetic skin creams.

The composition may also be administered or delivered to target cells in the form of liposomes. Liposomes are generally derived from phospholipids or other lipid substances and are formed by mono- or multi-lamellar hydrated liquid crystals that are dispersed in an aqueous medium. Specific examples of liposomes that may be used to administer or deliver a compound formula (I) include synthetic cholesterol, 1,2-distearoyl-sn-glycero-3-phosphocholine, 3-N-[(-methoxy poly(ethylene glycol)2000)carbamoyl]-1,2-dimyrestyloxy-propylamine (PEG-CDMA) and 1,2-di-o-octadecenyl-3-(N,N-dimethyl)aminopropane (DODMA).

The compositions may also be administered in the form of microparticles. Biodegradable microparticles formed from polylactide (PLA), polylactide-co-glycolide (PLGA), and \dot{ε}-caprolactone have been extensively used as drug carriers to increase plasma half life and thereby prolong efficacy (R. Kumar, M., 2000, J Pharm Pharmaceut Sci. 3(2) 234-258).

The compositions may incorporate a controlled release matrix that is composed of sucrose acetate isobutyrate (SAIB) and organic solvent or organic solvent mixtures. Polymer additives may be added to the vehicle as a release modifier to further increase the viscosity and slow down the release rate. The active compounds as described herein may be added to the SAIB delivery vehicle to form SAIB solution or suspension compositions. When the formulation is injected subcutaneously, the solvent diffuses from the matrix allowing the SAIB-drug or SAIB-drug-polymer mixtures to set up as an in situ forming depot.

In the treatment of HIV infection, therapeutic advantages may be obtained through combination treatment regimens as proposed herein. As such, methods of treatment according to the present invention may be used in conjunction with other therapies.

The co-administration of the active compounds as described herein may be simultaneous or sequential. Simultaneous administration may be effected by methods wherein the active compounds as described herein are in the same unit dose, or wherein the active compounds as described herein may be present in individual and discrete unit doses administered at the same, or at a similar time. Sequential administration may be in any order as required.

In another aspect, the present invention provides a method of screening for a compound that activates latent retrovirus infected cells, comprising contacting retrovirus target cells with a test compound and assessing whether said test compound activates the Wnt pathway in said target cells.

In another aspect, the present invention provides a method to establish the therapeutic dose for a patient infected with a retrovirus, of a test compound that activates latent retrovirus infected cells, comprising contacting retrovirus target cells of said patient with a test compound and assessing whether said test compound activates the Wnt pathway in said target cells.

In yet another aspect, the present invention provides a method to monitor a patient receiving treatment with a compound that activates latent retrovirus infected cells, said method comprising contacting retrovirus target cells from said patient with a test compound and assessing whether said test compound activates the Wnt pathway in said target cells.

Retroviruses infect specific target cells, this is called tropism. The methods for screening, monitoring and establishing a therapeutic dose, use the target cells of the retrovirus. For example HIV infects CD4+ memory T cells. Human T cell leukemia virus, (HTLV) has been found in CD4+ T cells, and other cell types in the peripheral blood including CD8+ T cells, dendritic cells and B cells. The methods described above, quantitate the activation of the Wnt pathway to monitor if sufficient activation is achieved by the doses of activators administered. Adjustment of doses can then be established ex vivo. One can asses whether a lower level of activator of the Wnt path is still sufficient, or whether an increase in the dose is required. The level of activation of Wnt target genes can be seen as an indicator of (to monitor) activation of the latent LTR.

In a preferred embodiment of the present invention and/or embodiments thereof, the retrovirus is HIV. In a preferred embodiment of the present invention and/or embodiments thereof, the target cell is a CD4+ memory T cell. The target cell may be a non-infected cell or a retrovirus infected cell.

In a preferred embodiment of the present invention and embodiments thereof, the compounds that activate latent retroviral infected cells, are activators of the Wnt pathway.

In a preferred embodiment of the present invention and embodiments thereof, the method for screening and/or for establishing the therapeutic dose for a patient, and/or monitoring a patient receiving treatment with a compound that activates latent retrovirus infected cells, comprises a test that measures Wnt pathway activation. As described above, a skilled person is well aware of several methods to determine activation of Wnt pathway.

All publications mentioned in this specification are herein incorporated by reference. The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

The present invention will now be further described in greater detail by reference to the following specific examples, which should not be construed as in any way limiting the scope of the invention.

### EXAMPLES

### Example 1

### Novel Transcription Regulatory Mechanism of Latent HIV LTR: Role of Wnt Pathway Activation on LTR Nucleosome Structure

The Wnt pathway is a highly conserved signaling pathway controlling a variety of biological processes. In the absence of Wnt, b-catenin is continuously degraded by the cytoplasmic destruction complex (FIG. 2). The destruction complex, composed of the core subunits APC, Axin1, CK1 and GSK3, binds and phosphorylates b-catenin, followed by its ubiquitination and proteosomal degradation. In the presence of Wnt, ubiquitination of phosphorylated b-catenin is blocked, the complex becomes saturated with phosphorylated b-catenin and functionally inactivated. Newly synthesized b-catenin then accumulates and is transported to the nucleus where it complexes with TCF/LEF to activate target genes (FIG. 2).

Interestingly, the HIV LTR harbours a TCF/LEF site within DNase hypersensitive site 1 (DHS1) within the LTR (FIG. 2). TCF/LEF transcription factors together with b-catenin constitute the molecular effectors of Wnt signaling. Given the presence of TCF/LEF sites within the HIV LTR, the role of Wnt pathway activation in de-repression of latent HIV was investigated in more detail.

A combinatorial strategy based on formaldehyde crosslinking is used to examine DNA accessibility, histone occupancy and transcription factor recruitment to latent and Wnt-activated HIV LTR in J-Lat cells containing full length latent HIV genome (FIG. 4A).

It was found that Wnt pathway activation via treatment with the GSK3b inhibitors LiCl or BIO (6-bromoindirubin-3-oxime), which result in stabilization of b-catenin, leads to recruitment of b-catenin to LTR DHS1 and nuc-1 as shown by Chromatin immunoprecipitation (ChIP) (FIG. 4D). As a result, histone acetylation is increased on the LTR and Lef1, while present on DHS1 is further enriched in response to Wnt activation (FIG. 4D). LiCl and BIO also increase accessibility concomitant with decreased nucleosome occupancy at DHS1 (FIG. 4B-C).

The high resolution dynamic nucleosomal structure of latently integrated HIV LTR is mapped in presence or absence of Wnt stimulation.

Using PCR-based MNase mapping (Rafati et al., 2011 PLos Biology 9(11) e1001206), it was found that DHS1 is not devoid of nucleosomes as previously thought but contains loosely positioned nucleosomes, which disappear upon PMA stimulation. It was further found that Wnt stimulation causes eviction of the loosely positioned nucleosome over DHS1 but does not affect nuc-1. Co-treatment with Valproic acid (VPA), a class I/II histone deacetylase inhibitor, however, causes a dramatic remodelling of the repressive nuc-1. It is an aspect of this invention that this synergistic effect on LTR nucleosome structure extends to other LTR de-repressors.

### Example 2

### Purging Latent HIV Using Small Molecules/Growth Factors Leading to Transcriptional Activation of Latent LTR

The role of Wnt pathway activation on LTR transcription is examined. The ligand R-spondin was recently shown to accentuate Wnt signaling (De Lau et al. 2011, Nature 476(7360):293-7).

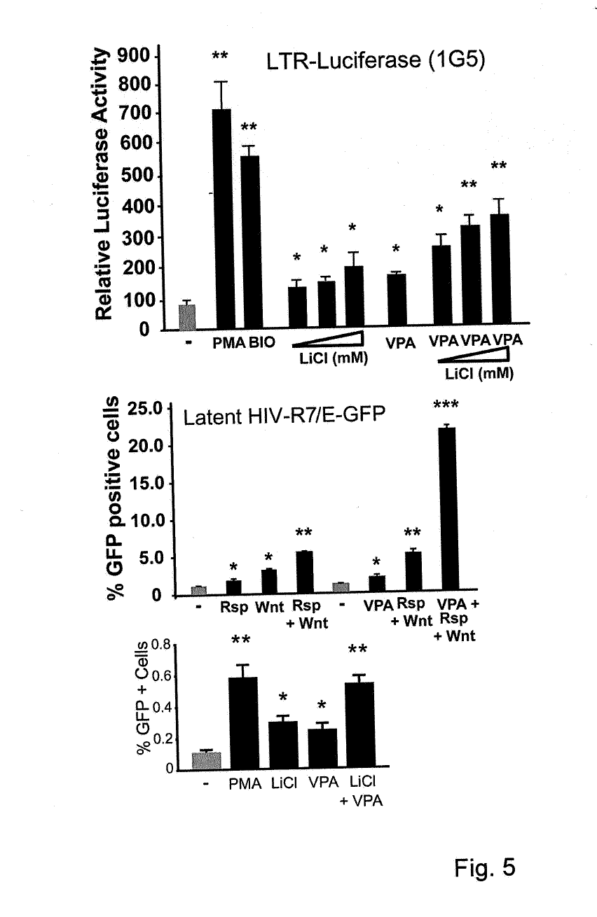

It was presently found that activation of the Wnt pathway in J-Lat/S-Lat or 1G5 cells via treatment with the ligands Wnt3A or R-spondin alone (or together synergistically), as well as the GSK3b inhibitors LiCl and BIO, resulted in de-repression of latent HIV LTR (FIG. 5).

The middle panel of FIG. 5 indicates the synergistic activation by using a Wnt3a-conditioned medium (medium from cell line with ATCC accession number CRL-2647) and a R-spondin1-conditioned medium (medium from cell line Human embryonic kidney cell-line 293T (HEK293T)) containing a stable integration of the R-spondin gene that express and secrete R-spondin (such a cell line can be obtained commercially from R&D Systems Inc. (Minneapolis, Minn., USA) or PeproTech Inc. (Rocky Hill, N.J., USA)). The growth medium was collected and used for activation experiments. The control cell-line is the same cell-line without the integration.

Strikingly, strong synergism in activation of latent LTR was found when the Wnt pathway is activated in the presence of VPA, which alone displayed marginal activation (FIG. 5). The functional synergism between Wnt activation and VPA was also evident when examining the LTR nucleosome structure; together they cause a drastic reduction is nucleosome density over the repressive nuc-1 concomitant with an increase in DNA accessibility resulting in removal of the positioned repressive nuc-1 (FIG. 4). Importantly, when examining the potential to purge latent infections established in Jurkat cells, cotreatment of cells with LiCl together with VPA resulted in strong synergism and re-activated latently infected cells to the same extent as treatment with the potent activator PMA (FIG. 5 bottom panel).

It is an aspect of this invention that the observed depletion of latent infections extends to other molecules implicated in LTR regulation such as DNA methylase inhibitors, prostratin, and 11-7.

### Example 3

### Primary Model Systems of HIV Latency

While Jurkat and SupT1 cells are a good well-established model for studying HIV transcription, they do not fully complement normal CD4+ T cells. A latency model in primary activated peripheral blood mononuclear cells (PBMCs) or purified CD4+ primary Tcells was therefore established.

PBMCs were infected with VSV-G pseudotyped LTR-Tat-IRES-GFP virus particles and examined establishment of a latent phenotype, as in the cell line latency models (J-Lat/S-Lat). Briefly, PBMCs were Ficoll-purified from buffycoats of healthy donors, activated with phytohemagglutinin (PHA-L) and Interleukin-2 (IL-2), followed by virus infection. Five days after infection, GFP negative cells were sorted by FACS, placed in culture. Importantly, treatment of these cells with PMA or TNFa activated HIV expression, identifying the presence of a population of latently infected cells similar to what was observed in J-Lat/S-Lat. Thus, even in the context of a fully activated T cell, integration of HIV at select sites will lead to the suppression of HIV transcription.

For this reason, the J-Lat/S-Lat latency establishment protocol allows direct transposition to primary human cells.

### Example 4

### Activation by? Lithium of Wnt Pathway but not NF-κB

In the present example it is shown that lithium does not activate the NFkb pathway at concentrations in which it activates the Wnt pathway and the HIV LTR. Zoltan H. Nemeth et al., published a manuscript entitled “Lithium Induces NFkB Activation and Interleukin-8 Production in Human Intestinal Epithelial Cells” Journal of Biological Chemistry, 2002, in which they show that Lithium when used in the concentration range between 20 mM-140 mM activates the NfKb pathway in the Human colon cancer cell lines HT-29 and CaCo-2.

Here we show that Lithium at 1 mM-10 mM concentration range, activates the Wnt pathway, and not the NFkB pathway in CD4+ T cell lines Jurkat and SupT1 as well as in primary CD4+ T cells and primary memory CD4+ T cell populations. RT-PCR analysis indicates that Lithium, when added to cells in the 1 mM-10 mM concentration range results in an increase in mRNA expression levels of the endogenous Wnt target genes AXIN2, TCF7(TCF1), MMP9, ZCCHC12, and MMP2, but not the NFkB target genes TLR2 or ICAM1. As a positive control, treatment of cells with Prostratin, a known activator of the NFkB pathway results in the activation of the NFkB target genes TLR2 and ICAM, see FIG. 9.

Therefore, addition of Lithium, in the 1-10 mM concentration range does not result in activation of the NFkB pathway, while the Wnt pathway is efficiently activated.

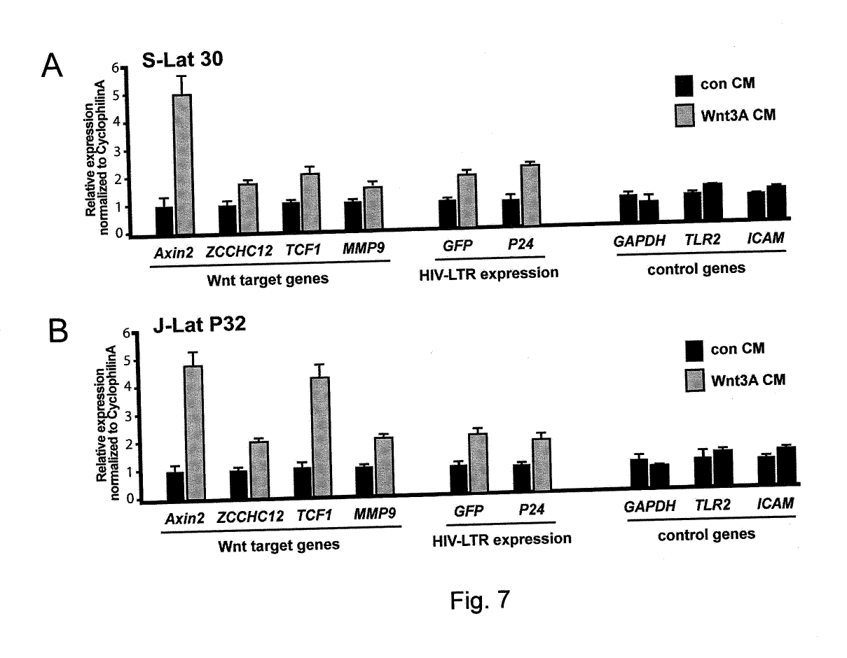

In addition, latent HIV-infected S-Lat 30 (A) and J-LatP32 (B) were treated with Wnt3A or control conditioned media. Relative mRNA expression levels of endogenous Wnt target genes AXIN2, ZCCHC12, TCF1, MMP9, the HIV P24 Gag and the LTR-reporter GFP, control (GAPDH) and NFkB target genes were quantitated by qRT-PCR. Expression data are presented as fold induction normalized to Cyclophillin A control. FIG. 7 shows that treatment of latent HIV infected cell with Wnt 3A ligand activates latent HIV and endogenous Wnt target genes but not NFkB target genes.

### Example 5

### Other Activators of Wnt Pathway

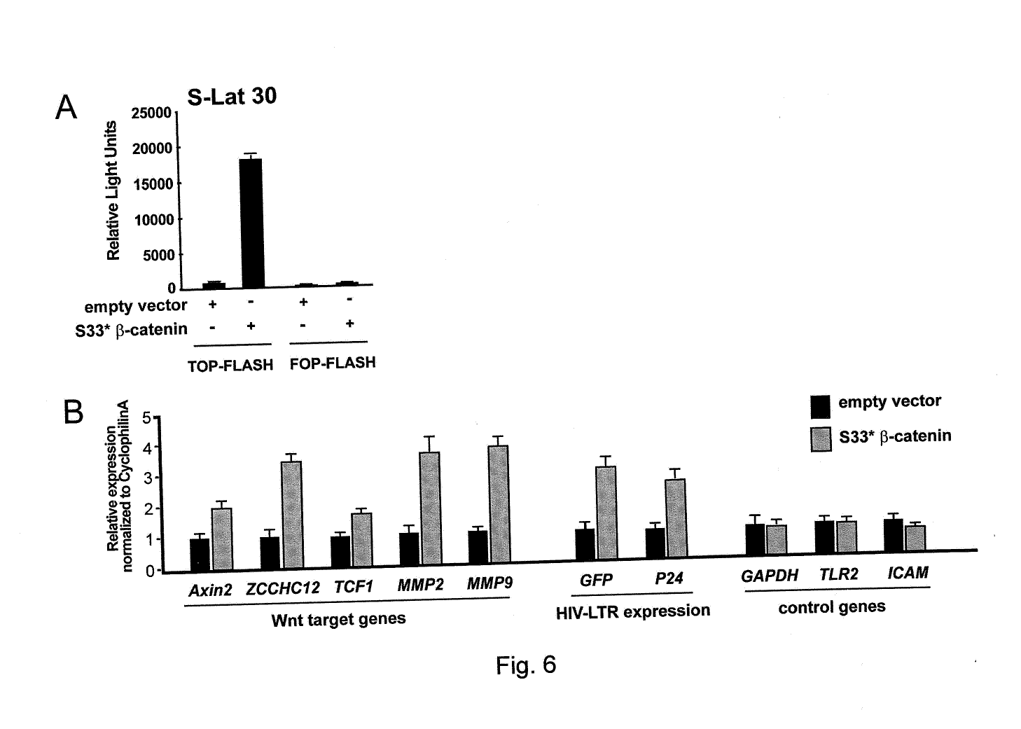

S33Y β-catenin constitutively active mutant: Transfection (by nucleofection) of latent HIV infected cell lines with an expression vector for a constitutively active form of β-catenin, the molecular effector of the Wnt signaling pathway results in activation of latent HIV. Exogenous expression of S33Y β-catenin in S-Lat30 results in activation of TCF-driven TOPFLASH luciferase activity, as well as increased mRNA expression of the endogenous Wnt target genes AXIN2, MMP2, TCF1, ZCCHC12, MMP9, but not control gene GAPDH, or the NFkb target genes TLR2 and ICAM1. TCF-driven activation of the Wnt pathway by S33Y β-catenin also results in a concomitant activation of latent HIV as quantitated by increased mRNA expression of HIV P24 Gag as well as the LTR-reporter GFP, see FIG. 6.

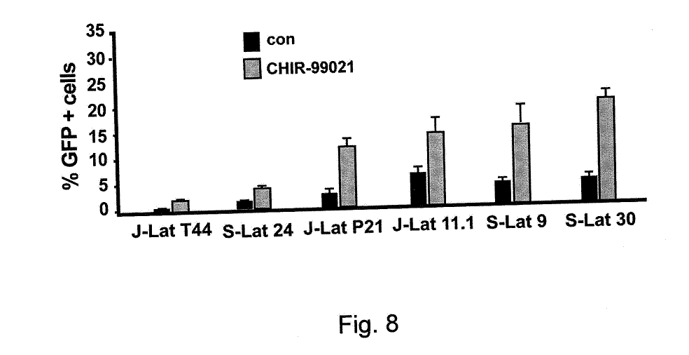

CHIR-99021: Treatment of a panel of CD4+ T J-Lat and S-Lat latent HIV-infected cell lines with the GSK3beta inhibitor CHIR-99021 results in activation of the HIV LTR, see FIG. 8. J-Lat P44, S-Lat 24, J-Lat P21, J-Lat 11.1, S-Lat 9 and S-Lat 30 latently infected Jurkat and SupT1 cell lines were treated with CHIR-99021 (1 μM) for 24 hours, and GFP expression was monitored by FACS analysis to determine activation of the latent LTR.

### Example 6

### Lithium and SAHA Activation

S-Lat 9, S-Lat 30, and J-Lat T44 harbouring a latently infected HIV-derived LTR-Tat-IRES-GFP virus, and J-Lat 11.1, which contains an integrated full length HIV virus harbouring GFP in place of Nef, were either untreated or treated with 2, 5, 10 mM LiCl, 500 uM SAHA alone or in combination. GFP expression was monitored by FACS analysis and is expressed as % GFP positive cells. Expression of GFP, P24, and the Wnt target genes AXIN2, MMP9, and TCF1 was monitored by qRT-PCR as indicated 72 hours after treatment. Expression data are presented as fold induction normalized to Cyclophillin A control, see FIG. 10.

### Example 7

### Lithium Treatment of Primary Cells

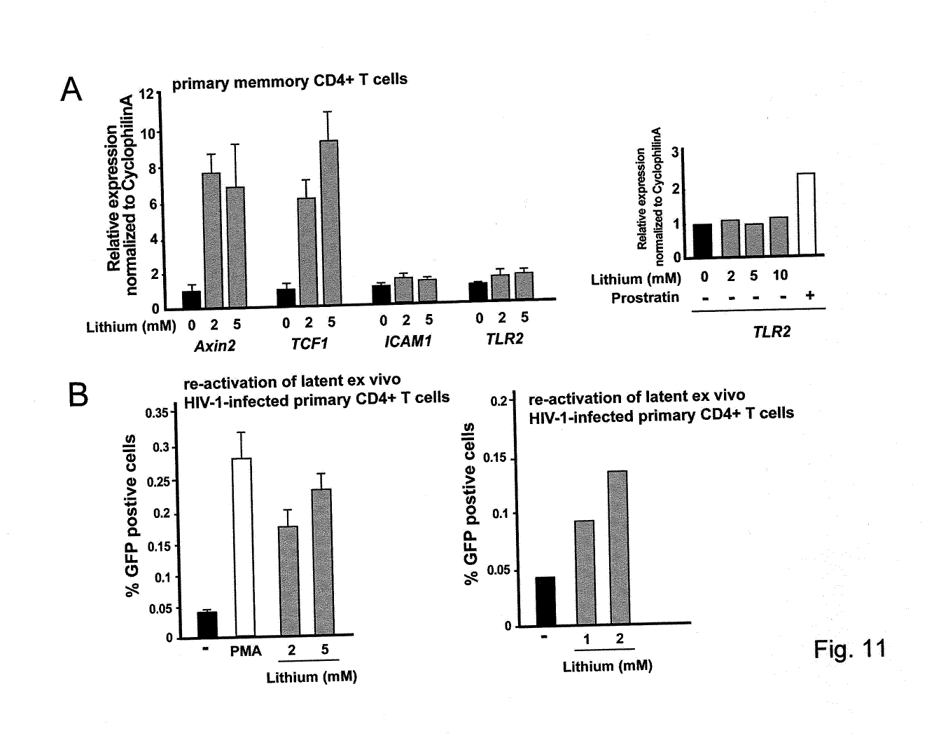

Lithium treatment activates Wnt and not NFkB target genes in memory CD4+ T cells. Memory CD4+ T cells were untreated or treated with Lithium or prostratin. mRNA expression of Wnt target genes AXIN2 and TCF1, as well as NFkB target genes TLR2 and ICAM1 was monitored by qRT-PCR 15 hours after treatment. Primary CD4+ T cells were purified from buffy coats from healthy donors and infected ex-vivo with HIV-derived retroviral vector LTR-Tat-IRES-GFP. GFP negative cells comprising uninfected and latently infected cells were sorted by flow cytometry and treated with Lithium (1-5 mM). GFP expression was monitored by FACS analysis to determine activation of the latent HIV LTR. See FIG. 11.

1-29. (canceled)

30. A method of treating a subject infected with a retrovirus, said method comprising administering to said subject an activator of the Wnt pathway in an amount that increases Wnt pathway signalling in a retrovirus target cell of said subject such that the retroviral long terminal repeat (LTR) in said cells is activated or de-repressed.

31. The method according to claim 30, wherein in the event that lithium is used to increase Wnt pathway signalling, the amount of lithium administered provides a plasma concentration in said subject between 0.1-10 mM.

32. The method of claim 30, wherein said retrovirus is HIV virus, preferably HIV-1, and/or wherein said cell is a CD4+ T cell, preferably a resting memory CD4+ T cell.

33. The method according to claim 30, wherein said subject receives antiretroviral therapy.

34. The method of claim 30, wherein said activator is selected from Wnt3A, R-spondin, 6-Bromoindirubin-3′-oxime (BIO), 6-(2-(4-(2,4-Dichlorophenyl)-5-(4-methyl-1H-imidazol-2-yl)-pyrimidin-2-ylamino)ethyl-amino)-nicotinonitrile, and lithium, and combinations thereof.

35. The method of claim 30, wherein said activator is administered in combination with a histone deacetylase inhibitor (HDACi), preferably valproic acid (VPA) and/or vorinostat (SAHA).

36. The method of claim 30, wherein said method further comprises administering a compound selected from a cytokine, a T cell activation signal, a macrophage activator, a protein kinase C (PKC) activator, a nuclear factor kB (NF-kB) activator, a transcription elongation inducer, and combinations thereof.

37. A method for increasing retrovirus transcription in an infected eukaryotic cell, comprising increasing Wnt pathway signaling in said cell such that transcription of said retrovirus is increased.

38. The method of claim 37, wherein said infected cell is a latently infected cell wherein retrovirus transcription is repressed, said method comprising activating said repressed retrovirus by increasing Wnt pathway signaling in said cell such that the retroviral long terminal repeat (LTR) is activated or de-repressed.

39. The method of claim 37, wherein Wnt pathway signaling in said cell is increased such that beta-catenin is stabilized and recruited to the retroviral long terminal repeat (LTR).

40. The method of claim 37, wherein Wnt pathway signaling in said cell is increased by contacting said cell with a Wnt pathway activator, an inhibitor of glycogen synthase kinase-3 beta (GSK-3β), an inhibitor of adenomatous polyposis coli (APC), or an inhibitor of axin, or a combination thereof.

41. The method of claim 37, wherein said GSK-3β inhibitor is (6-bromoindirubin-3′-oxime (BIO) and/or lithium.

42. The method of claim 41, wherein said Wnt pathway activator is a Wnt ligand, such as Wnt3A, and/or an LGR ligand, such as R-spondin.

43. The method of claim 37 wherein in the event that lithium is used to increase Wnt pathway signalling, the amount of lithium is between 0.1-10 mM.

44. The method of claim 37, wherein said method further comprises increasing histone acetylation in said cell.

45. The method of claim 44, wherein said histone acetylation is increased by contacting said cell with a histone deacetylase inhibitor (HDACi).

46. The method of claim 45, wherein said HDACi is valproic acid (VPA) and/or vorinostat (SAHA), and wherein Wnt signalling is increased by contacting said cell with the GSK-3β inhibitor lithium.

47. The method of claim 37, wherein said method further comprises contacting said cell with a compound selected from a cytokine, a T cell activation signal, a macrophage activator, a protein kinase C (PKC) activator, a nuclear factor kB (NF-kB) activator, a transcription elongation inducer, and combinations thereof.

48. The method of claim 47, wherein said compound is selected from the group consisting of LPS, Il-7, prostratin, hexamethylbisacetamide (HMBA), and CyclinT.

49. The method of claim 37, wherein said retrovirus is the human immunodeficiency virus (HIV), preferably HIV-1.

50. The method of claim 38, wherein said LTR is the 5′-LTR.

51. The method of claim 37, wherein said cell is a CD4+ T cell, preferably a resting memory CD4+ T cell.

52. A method of establishing the therapeutic dose, for a patient that has been infected by a retrovirus, of a test compound that activates latent retrovirus infected cells, comprising contacting retrovirus target cells of said patient with a test compound and assessing whether said test compound activates the Wnt pathway in said target cells.

53. A method of monitoring a patient receiving treatment with a compound that activates latent retrovirus infected cells, said method comprising contacting retrovirus target cells from said patient with a test compound and assessing whether said test compound activates the Wnt pathway in said target cells.

54. Method according to any of claims 51-53 wherein said retrovirus is HIV, preferably HIV-1, and/or wherein said target cell is a CD4+ T cell, preferably a resting memory CD4+ T cell.

55. Method according to any of claims 51-53 wherein said method comprises a test that measures Wnt pathway activation.

56. Method according to any of claims 51-53 wherein said compound that activates latent retrovirus infected cells is an activator of the Wnt pathway.
